(19) United States
(12) Patent Application Publication
(10) Pub. No.: US 2017/0360978 A1
(43) Pub. Date: Dec. 21, 2017

(54) AQUEOUS PERFUME COMPOSITIONS AND FRESHENING PRODUCTS COMPRISING THE AQUEOUS PERFUME COMPOSITIONS CONTAINED IN PRESSURIZED PLASTIC CONTAINERS

(71) Applicant: The Procter & Gamble Company, Cincinnati, OH (US)

(72) Inventors: Chisomaga Ugochi NWACHUKWU, Cincinnati, OH (US); Andrew William FRANCKHAUSER, Batavia, OH (US); Nicole Lynette KATTAU, Mason, OH (US); Zaiyou LIU, West Chester, OH (US); John Andrew MCDANIEL, Middletown, OH (US); Christine Marie READNOUR-GOUGE, Ft. Mitchell, KY (US); Judith Ann HOLLINGSHEAD, Batavia, OH (US)

(21) Appl. No.: 15/627,479

(22) Filed: Jun. 20, 2017

Related U.S. Application Data
(60) Provisional application No. 62/352,151, filed on Jun. 20, 2016.

(51) Int. Cl. A61L 9/012; A61L 9/14; B65D 83/38; B65D 83/14

(52) U.S. Cl. CPC A61L 9/012; B65D 83/752; A61L 9/14; B65D 83/38

(57) ABSTRACT

A freshening product is provided. The freshening product includes a pressurized plastic container having an internal gage pressure of about 414 kPa to about 1100 kPA and an aqueous freshening composition having: at least one controlled perfume raw material (“Controlled PRM”) selected from the group consisting of: (z)-1-((1r,2s)-2,6,6-trimethylcyclohex-3-en-1-yl)but-2-en-1-one; 3-(2-ethylphenyl)-2,2-dimethylpropanal; 3,7-dimethyloct-6-en-1-ol; 3-(4-isopropylphenyl)-2-methylpropanal; 5-heptyldihydrofuran-2(3h)-one; (e)-4-methyldec-3-en-5-ol; 4-(tert-butyl)cyclohexyl acetate; 2-(tert-butyl)cyclohexyl acetate; allyl hexanoate; 2,6-dimethyloct-7-en-2-ol; 2-methyl-1-phenylpropan-2-yl acetate; (z)-cyclooct-4-en-1-yl methyl carbonate; (r)-1-methyl-4-(prop-1-en-2-yl)cyclohex-1-ene; 3,7-dimethylocta-1,6-dien-3-ol; 3,7-dimethylocta-1,6-dien-3-yl acetate; 3-(4-(tert-butyl)phenyl)-2-methylpropanal; cyclohexyl acetate; p-cymene; (e)-3,7-dimethylnona-1,6-dien-3-ol; (e)-4-(2,6,6-trimethylcyclohex-1-en-1-yl)but-3-en-2-one; (e)-4-(2,6,6-trimethylcyclohex-2-en-1-yl)but-3-en-2-one; (z)-3-methyl-4-(2,6,6-trimethylcyclohex-2-en-1-yl)but-3-en-2-one; 1-(2,6,6-trimethyl-1-cyclohex-2-en-1-yl)pent-1-en-3-one; 3a,4,5,6,7,7a-hexahydro-4,7-methano-1(3)h-inden-6(1)-yl propanoate; 3a,4,5,6,7,7a-hexahydro-4,7-methano-1(3)h-inden-6(1)-yl acetate; ethyl 2-(2-methyl-1,3-dioxolan-2-yl)acetate; and combinations thereof, or at least one limited perfume raw material (“Limited PRM”) selected from the group consisting of: ethyl 2-methylbutanoate; 3,5,5-trimethylhexyl acetate; ethyl 2-methylpentanoate; propyl (s)-2-(tert-pentyloxy)propanoate; 1-(3,3-dimethylcyclohexyl)ethyl formate, 4-methylquinoline; 1-heptanol; and combinations thereof, wherein each Controlled PRM or each Limited PRM, when present, is present at a level up to 0.3%, by weight of the aqueous freshening composition.

### FIELD

The present disclosure relates to improved aqueous freshening compositions and freshening products comprising the aqueous freshening compositions contained in pressurized plastic containers.

### BACKGROUND

Pressurized containers for dispensing freshening compositions are known in the art and are typically constructed of metal in order to withstand the internal pressure of aerosols. Pressurized plastic containers containing a freshening composition may be desirable for cost, aesthetic (e.g. clear container), and recyclability advantages. It has been found, however, that the interaction of the perfume mixtures present in freshening compositions with plastic containers, especially when under pressure, may result in a phenomenon called crazing. Crazing is the appearance of small cleaves in the plastic, resembling cracks. Crazing is believed to be affected by the morphology of the plastic container, the strain on the container, and/or the chemistry of the composition contained in the container. Crazing is undesirable from both an aesthetic and functional point of view.

Manufacturers have attempted many techniques to minimize/avoid crazing in pressurized plastic containers due to certain chemical interactions of the formulations contained therein. These techniques include adjusting geometries and thicknesses of the container wall, applying a coating layer to the container wall, crystallizing certain portions of the container, and adjusting certain formulation chemistries. Attempts to overcome crazing problems are shown, for example, in U.S. Pat. No. 7,303,087 and WO 2011/088093. However, these approaches have not proven entirely satisfactory where a perfume mixture may be the primary active in the formulation (e.g. air freshening sprays). Further, some of the previous approaches require added production steps and/or costs (e.g. crystallizing neck portions, adding coatings, and constructing thicker walls than required for safety) and may be viewed as environmentally unfriendly.

As such, there remains a need for improved aqueous freshening compositions that minimize crazing in pressurized plastic containers.

### SUMMARY

Examples/Combinations
A. A freshening product comprising:

a pressurized plastic container having an internal gage pressure of about 414 kPa to about 1100 kPA; and

an aqueous freshening composition comprising:

(a) at least one controlled perfume raw material (“Controlled PRM”) selected from the group consisting of: (z)-1-((1r,2s)-2,6,6-trimethylcyclohex-3-en-1-yl)but-2-en-1-one; 3-(2-ethylphenyl)-2,2-dimethylpropanal; 3,7-dimethyloct-6-en-1-ol; 3-(4-isopropylphenyl)-2-methylpropanal; 5-heptyldihydrofuran-2(3h)-one; (e)-4-methyldec-3-en-5-ol; 4-(tert-butyl)cyclohexyl acetate; 2-(tert-butyl)cyclohexyl acetate; allyl hexanoate; 2,6-dimethyloct-7-en-2-ol; 2-methyl-1-phenylpropan-2-yl acetate; (z)-cyclooct-4-en-1-yl methyl carbonate; (r)-1-methyl-4-(prop-1-en-2-yl)cyclohex-1-ene; 3,7-dimethylocta-1,6-dien-3-ol; 3,7-dimethylocta-1,6-dien-3-yl acetate; 3-(4-(tert-butyl)phenyl)-2-methylpropanal; cyclohexyl acetate; p-cymene; (e)-3,7-dimethylnona-1,6-dien-3-ol; (e)-4-(2,6,6-trimethylcyclohex-1-en-1-yl)but-3-en-2-one; (e)-4-(2,6,6-trimethylcyclohex-2-en-1-yl)but-3-en-2-one; (z)-3-methyl-4-(2,6,6-trimethylcyclohex-2-en-1-yl)but-3-en-2-one; 1-(2,6,6-trimethyl-1-cyclohex-2-en-1-yl)pent-1-en-3-one; 3a,4,5,6,7,7a-hexahydro-4,7-methano-1(3)h-inden-1-yl propanoate; 3a,4,5,6,7,7a-hexahydro-4,7-methano-1H-inden-6-yl propionate; 3a,4,5,6,7,7a-hexahydro-1H-4,7-methanoinden-6-yl acetate; 3a,4,5,6,7,7a-hexahydro-1H-4,7-methanoinden-1-yl acetate; 3a,4,5,6,7,7a-hexahydro-4,7-methano-1H-inden-5-yl acetate; ethyl 2-(2-methyl-1,3-dioxolan-2-yl)acetate; and combinations thereof; or

(b) at least one limited perfume raw material (“Limited PRM”) selected from the group consisting of: ethyl 2-methylbutanoate; 3,5,5-trimethylhexyl acetate; ethyl 2-methylpentanoate; propyl (s)-2-(tert-pentyloxy)propanoate; 1-(3,3-dimethylcyclohexyl)ethyl formate, 4-methylquinoline; 1-heptanol; and combinations thereof,
wherein each Controlled PRM or each Limited PRM, when present, is present at a level up to 0.3%, by weight of the aqueous freshening composition.
B. The freshening product of PARAGRAPH A, wherein the total level of Controlled PRMs present in the aqueous freshening composition is 0.6 wt. % or less, by weight of the aqueous freshening composition, and wherein the total level of Limited PRMs present in the aqueous freshening composition is 0.1 wt. % or less, by weight of the aqueous freshening composition.
C. The freshening product of PARAGRAPH A OR B, wherein the aqueous freshening composition comprises at least one moderately compatible perfume raw material “(Moderately Compatible “PRM”) selected from the group consisting of: benzyl acetate; oxydibenzene; 3-(benzo[d][1,3]dioxol-5-yl)-2-methylpropanal; 4-(4-hydroxy-4-methylpentyl)cyclohex-3-ene-1-carbaldehyde; hexyl 2-hydroxybenzoate; 1,4-dioxacycloheptadecane-5,17-dione; (Z)-hex-3-en-1-yl acetate; 1-phenylethyl acetate; 2-((3,7-dimethyloct-6-en-1-yl)oxy)acetaldehyde; 2-phenoxyethyl isobutyrate; (Z)-2-benzylideneoctanal; 2,4-dimethylcyclohex-3-ene-1-carbaldehyde; 2,6-dimethylhept-5-enal; methyl 2-(3-oxo-2-pentylcyclopentyl)acetate; benzyl methyl ether; benzyl ether; 4-methylbenzaldehyde; 3,5,5-trimethyl-2-cyclohexen-1-one; and combinations thereof, wherein each Moderately Compatible PRM, when present, is present at level up to 1.0 wt. %, preferably up to 0.6 wt. %, by weight of the aqueous freshening composition.
D. The freshening product ANY OF PARAGRAPHS A THROUGH C, wherein the pressurized plastic container comprises a hoop tensile strain of 0.1% to 2%, preferably from 0.2% to 0.6%.
E. The freshening product of ANY OF PARAGRAPHS A THROUGH D, wherein the aqueous carrier is present in an amount greater than 70 wt. %, preferably greater than 80% to 99.9%, more preferably greater than 90% to 99.9% by weight of the aqueous freshening composition.
F. The freshening product of ANY OF PARAGRAPHS A THROUGH E, wherein the perfume mixture is present in the aqueous freshening composition in an amount of 0.01 wt. % to 10 wt. %, by weight of the aqueous freshening composition.
G. The freshening product of ANY OF PARAGRAPHS A THROUGH F, further comprising a propellant selected from the group consisting of: nitrogen, carbon dioxide, compressed air, and mixtures thereof.
H. The freshening product of ANY OF PARAGRAPHS A THROUGH G, wherein the pressurized plastic container comprises a bag-in-bottle container comprising a hydrofluoro olefin propellant.
I. The freshening product of ANY OF PARAGRAPHS A THROUGH H, wherein said propellant is free of hydrocarbons.
J. The freshening product of ANY OF PARAGRAPHS A THROUGH I, further comprising a solubilizer selected from group consisting of: non-ionic surfactant, alcohol, glycol, and mixtures thereof.
K. The freshening product of ANY OF PARAGRAPHS A THROUGH J, further comprising 1-((2S,3S)-2,3,8,8-tetramethyl-1,2,3,4,5,6,7,8-octahydronaphthalen-2-yl)ethan-1-one; or benzyl 2-hydroxybenzoate.
L. A freshening product comprising:

a pressurized plastic container; and

an aqueous freshening composition comprising at least one highly compatible perfume raw material (“Highly Compatible PRM”) selected from the group consisting of: 2-isobutyl-4-methyltetrahydro-2H-pyran-4-ol; (3aR,5aR,9aR,9bR)-3a,6,6,9a-tetramethyldodecahydronaphtho[2,1-b]furan; 1-methoxy-4-methylbenzene; 4,6,6,7,8,8-hexamethyl-1,3,4,6,7,8-hexahydrocyclopenta[g]isochromene; 4-methoxybenzaldehyde; phenylmethanol; 3-methylbut-2-en-1-yl acetate; benzaldehyde; 2-phenylethan-1-ol; 2-(p-tolyloxy)acetaldehyde; ethyl butyrate; (E)-3-methylcyclopentadec-4-en-1-one; (E)-oxacyclohexadec-13-en-2-one, and combinations thereof.
M. The freshening product of PARAGRAPH L, wherein the pressurized plastic container is pressurized to an internal gage pressure of 414 kPa to 1100 kPA, preferably 460 kPa to 1100 kPa, more preferably 550 kPa to 1100 kPa, most preferably 827 kPa to 1100 kPa.
N. The freshening product of PARAGRAPH L OR PARAGRAPH M, wherein the aqueous freshening composition comprises at least two Highly Compatible PRMs.
O. The freshening product of ANY OF PARAGRAPHS L THROUGH N, wherein the aqueous freshening composition comprises at least one moderately compatible perfume raw material “(Moderately Compatible “PRM”) selected from the group consisting of: benzyl acetate; oxydibenzene; 3-(benzo[d][1,3]dioxol-5-yl)-2-methylpropanal; 4-(4-hydroxy-4-methylpentyl)cyclohex-3-ene-1-carbaldehyde; hexyl 2-hydroxybenzoate; 1,4-dioxacycloheptadecane-5,17-dione; (Z)-hex-3-en-1-yl acetate; 1-phenylethyl acetate; 2-((3,7-dimethyloct-6-en-1-yl)oxy)acetaldehyde; 2-phenoxyethyl isobutyrate; (Z)-2-benzylideneoctanal; 2,4-dimethylcyclohex-3-ene-1-carbaldehyde; 2,6-dimethylhept-5-enal; methyl 2-(3-oxo-2-pentylcyclopentyl)acetate, benzyl methyl ether; benzyl ether; 4-methylbenzaldehyde; 3,5,5-trimethyl-2-cyclohexen-1-one; and combinations thereof, wherein each Moderately Compatible PRM, when present, is present at level up to 1.0 wt. %, more preferably up to 0.6 wt. %, by weight of the aqueous freshening composition.
P. The freshening product of PARAGRAPH O, wherein the total level of Moderately Compatible PRMs present in the aqueous freshening composition is 1.2 wt. % or less, by weight of the aqueous freshening composition.
Q. The freshening product of ANY OF PARAGRAPHS L THROUGH P, wherein the aqueous freshening composition comprises at least one controlled perfume raw material (“Controlled PRM”) selected from the group consisting of: (z)-1-((1r,2s)-2,6,6-trimethylcyclohex-3-en-1-yl)but-2-en-1-one; 3-(2-ethylphenyl)-2,2-dimethylpropanal; 3,7-dimethyloct-6-en-1-ol; 3-(4-isopropylphenyl)-2-methylpropanal; 5-heptyldihydrofuran-2(3h)-one; (e)-4-methyldec-3-en-5-ol; 4-(tert-butyl)cyclohexyl acetate; 2-(tert-butyl)cyclohexyl acetate; allyl hexanoate; 2,6-dimethyloct-7-en-2-ol; 2-methyl-1-phenylpropan-2-yl acetate; (z)-cyclooct-4-en-1-yl methyl carbonate; (r)-1-methyl-4-(prop-1-en-2-yl)cyclohex-1-ene; 3,7-dimethylocta-1,6-dien-3-ol; 3,7-dimethylocta-1,6-dien-3-yl acetate; 3-(4-(tert-butyl)phenyl)-2-methylpropanal; cyclohexyl acetate; p-cymene; (e)-3,7-dimethylnona-1,6-dien-3-ol; (e)-4-(2,6,6-trimethylcyclohex-1-en-1-yl)but-3-en-2-one; (e)-4-(2,6,6-trimethylcyclohex-2-en-1-yl)but-3-en-2-one; (z)-3-methyl-4-(2,6,6-trimethylcyclohex-2-en-1-yl)but-3-en-2-one; 1-(2,6,6-trimethyl-1-cyclohex-2-en-1-yl)pent-1-en-3-one; 3a,4,5,6,7,7a-hexahydro-4,7-methano-1(3)h-inden-6(1)-yl propanoate; 3a,4,5,6,7,7a-hexahydro-4,7-methano-1(3)h-inden-6(1)-yl acetate; ethyl 2-(2-methyl-1,3-dioxolan-2-yl)acetate; and combinations thereof, wherein each Controlled PRM, when present, is present at level up to 0.3 wt. %, more preferably up to 0.1 wt. %, by weight of the aqueous freshening composition.
R. The freshening product of PARAGRAPH Q, wherein the total level of Controlled PRMs present in the aqueous freshening composition is 0.6 wt. % or less, by weight of the aqueous freshening composition.
S. The freshening product of ANY OF PARAGRAPHS L THROUGH R, wherein the aqueous freshening composition comprises at least one limited perfume raw material (“Limited PRM”) selected from the group consisting of: ethyl 2-methylbutanoate; 3,5,5-trimethylhexyl acetate; ethyl 2-methylpentanoate; propyl (s)-2-(tert-pentyloxy)propanoate; 1-(3,3-dimethylcyclohexyl)ethyl formate, 4-methylquinoline; 1-heptanol; and combinations thereof, wherein each Limited PRM, when present, is present at level up to 0.3 wt. %, more preferably up to 0.1 wt. %, more preferably up to 0.05 wt. %, most preferably up to 0.01 wt. %, by weight of the aqueous freshening composition.
T. The freshening product of PARAGRAPH S, wherein the total level of Limited PRMs present in the aqueous freshening composition is 0.1 wt. % or less, by weight of the aqueous freshening composition.
U. The freshening product ANY OF PARAGRAPHS L THROUGH T, wherein the pressurized plastic container comprises a hoop tensile strain of 0.1% to 2%, preferably from 0.2% to 0.6%.
V. The freshening product of ANY OF PARAGRAPHS L THROUGH U, wherein the aqueous carrier is present in an amount greater than 70 wt. %, preferably greater than 80% to 99.9%, more preferably greater than 90% to 99.9% by weight of the aqueous freshening composition.
W. The freshening product of ANY OF PARAGRAPHS L THROUGH V, wherein the perfume mixture is present in the aqueous freshening composition in an amount of 0.01 wt. % to 10 wt. %, by weight of the aqueous freshening composition.
X. The freshening product of ANY OF PARAGRAPHS L THROUGH W, further comprising a propellant selected from the group consisting of: nitrogen, carbon dioxide, compressed air, and mixtures thereof.
Y. The freshening product of ANY OF PARAGRAPHS L THROUGH X, wherein the pressurized plastic container comprises a bag-in-bottle container comprising a hydrofluoro olefin propellant.
Z. The freshening product of ANY OF PARAGRAPHS L THROUGH Y, wherein said propellant is free of hydrocarbons.
AA. The freshening product of ANY OF PARAGRAPHS L THROUGH Z, further comprising a solubilizer selected from group consisting of: non-ionic surfactant, alcohol, glycol, and mixtures thereof.
BB. The freshening product of ANY OF PARAGRAPHS L THROUGH AA, further comprising 1-((2S,3S)-2,3,8,8-tetramethyl-1,2,3,4,5,6,7,8-octahydronaphthalen-2-yl)ethan-1-one; or benzyl 2-hydroxybenzoate.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a fragmentary, frontal view of a container having a flange and lower neck region which is usable to calculate hoop tensile strain according to the present invention.

FIG. 2 is a fragmentary, vertical sectional view taken along lines 2-2 of FIG. 1.

FIG. 3 is an enlarged view of the neck region denoted with the boundary region 3 shown in FIG. 2.

FIG. 4 is a fragmentary vertical sectional view of a plastic container having an optional flange, and showing the respective axial midpoint MP of the neck of such plastic container.

FIG. 5 is a visual scale of % crazed for plastic test bars.

FIG. 6 is a plot of the perfume raw material crazing profile with plastic under strain.

### DETAILED DESCRIPTION

The present disclosure provides a freshening product comprising an aqueous freshening composition that minimizes crazing in a pressurized plastic container.

Aqueous Freshening Composition

The aqueous freshening composition of the present disclosure comprises a perfume mixture and an aqueous carrier. The final pH of the aqueous freshening composition herein may be from about 1 to about 11, alternatively from about 3 to about 10, alternatively from about 4 to about 8.

Perfume Raw Materials and Perfume Mixtures

The aqueous freshening compositions comprise a perfume mixture. The perfume mixture may comprise one or more perfume raw materials (“PRMs”). “Perfume” refers to organic substances that are included in a product to provide a desired olfactory property, whether scented, low-scent/unscented. A perfume may include chemicals that are aromatic, aliphatic, ionone, hydrocarbon, alcohol, aldehyde, ketone, and ester.

It has been found that perfume raw materials affect crazing of pressurized plastic containers to varying degrees. Specifically, it has been found that some perfume raw materials result in negligible crazing to a pressurized plastic container at any level, or up to a certain level. Some perfume raw materials result in minor crazing to pressurized plastic containers at low levels of the particular perfume raw materials. Other perfume raw materials result in minor, visible, or major crazing at any level of the particular perfume raw materials.

Highly Compatible PRMs

Perfume raw materials that have been found to provide negligible crazing to pressurized plastic containers at any level of the perfume raw material in the aqueous freshening composition may include perfume raw materials selected from the group consisting of the perfume raw materials in Table 1 or combinations thereof (“highly compatible PRMs”).

TABLE 1

| Highly | IUPAC Name for Highly | |
|---|---|---|
| Compatible PRMs | Compatible PRMs | CAS No |
| pyranol | 2-isobutyl-4-methyltetrahydro- | 63500-71-0 |
| | 2H-pyran-4-ol | |
| silvanone m | (3aR,5aR,9aR,9bR)-3a,6,6,9a- | 3738-00-9 |
| | tetramethyldodecahydronaphtho | |
| | [2,1-b]furan | |
| p-cresyl methyl ether | 1-methoxy-4-methylbenzene | 104-93-8 |
| (anisole) | | |
| hexamethylindanopyran | 4,6,6,7,8,8-hexamethyl- | 1222-05-5 |
| | 1,3,4,6,7,8- | |
| | hexahydrocyclopenta[g] | |
| | isochromene | |
| anisic aldehyde | 4-methoxybenzaldehyde | 123-11-5 |
| iso e super or wood | 1-((2S,3S)-2,3,8,8-tetramethyl- | 54464-57-2 |
| | 1,2,3,4,5,6,7,8- | |
| | octahydronaphthalen-2-yl)ethan- | |
| | 1-one | |
| benzyl salicylate | benzyl 2-hydroxybenzoate | 118-58-1 |
| benzyl alcohol | phenylmethanol | 100-51-6 |
| prenyl acetate | 3-methylbut-2-en-1-yl acetate | 1191-16-8 |
| benzaldehyde | benzaldehyde | 100-52-7 |
| phenyl ethyl alcohol | 2-phenylethan-1-ol | 60-12-8 |
| p-methyl phenoxy | 2-(p-tolyloxy)acetaldehyde | 67845-46-9 |
| acetaldehyde | | |
| ethyl butyrate | ethyl butyrate | 105-54-4 |
| delta muscenone | (E)-3-methylcyclopentadec-4-en- | 63314-79-4 |
| 962191 | 1-one | |
| habanolide 100% | (E)-oxacyclohexadec-13-en-2-one | 111879-80- |
| | | 2 |

Each of the Highly Compatible PRMs may be may be present at any level in the aqueous freshening composition. Preferably each of the Highly Compatible PRMs may be present in the aqueous freshening composition at a level up to 0.1 wt. %, or up to 0.5 wt. %, or up to 1.0 wt. %, or up to 2.0 wt. %, or up to 3.0 wt. %, or up to 4.0 wt. %, or up to 5.0 wt. %, or up to 6.0 wt. %, or up to 7.0 wt. %, or up to 8.0 wt. %, or up to 9.0 wt. %, or up to 10 wt. %, or up to 20 wt. %, or up to 30 wt. %, or up to 40 wt. %, or up to 50 wt. %, or up to 60 wt. %, or up to 70 wt. %, or up to 80 wt. %, or up to 90 wt. %, by weight of the aqueous freshening composition.

Moderately Compatible PRMs

Perfume raw materials that have been found to provide negligible or minor crazing to pressurized plastic containers in the aqueous freshening composition may be selected from the group consisting of the perfume raw materials listed in Tables 2A and 2B and combinations thereof (“Moderately Compatible PRMs”).

TABLE 2A

| | IUPAC Name for | |
|---|---|---|
| Moderately Compatible | Moderately Compatible | |
| PRMs | PRMs | CAS No |
| benzyl acetate | benzyl acetate | 140-11-4 |
| diphenyloxide | oxydibenzene | 101-84-8 |
| helional | 3-(benzo[d][1,3]dioxol-5- | 1205-17-0 |
| | yl)-2-methylpropanal | |
| lyral | 4-(4-hydroxy-4- | 31906-04-4 |
| | methylpentyl)cyclohex-3- | |
| | ene-1-carbaldehyde | |
| hexyl salicylate | hexyl 2-hydroxybenzoate | 6259-76-3 |
| ethylene brassylate | 1,4- | 105-95-3 |
| | dioxacycloheptadecane- | |
| | 5,17-dione | |
| cis-3-hexenyl acetate | (Z)-hex-3-en-1-yl acetate | 3681-71-8 |
| methyl phenyl carbinyl | 1-phenylethyl acetate | 93-92-5 |
| acetate | | |
| citronellyl oxyacetaldehyde | 2-((3,7-dimethyloct-6-en- | 7492-67-3 |
| | 1-yl)oxy)acetaldehyde | |
| phenoxy ethyl iso butyrate | 2-phenoxyethyl isobutyrate | 103-60-6 |
| hexyl cinnamic aldehyde | (Z)-2-benzylideneoctanal | 101-86-0 |
| ligustral or triplal | 2,4-dimethylcyclohex-3- | 68039-49-6 |
| | ene-1-carbaldehyde | |
| melonal | 2,6-dimethylhept-5-enal | 106-72-9 |
| methyl dihydro jasmonate | methyl 2-(3-oxo-2- | 24851-98-7 |
| | pentylcyclopentyl)acetate | |

TABLE 2B

| | IUPAC Name for | |
|---|---|---|
| Moderately Compatible | Moderately Compatible | |
| PRMs | PRMs | CAS No |
| Benzyl methyl ether | benzyl methyl ether | 538-86-3 |
| Dibenzyl ether | benzyl ether | 103-50-4 |
| p-Tolualdehyde | 4-methylbenzaldehyde | 104-87-0 |
| Isophorone | 3,5,5-trimethyl-2- | 78-59-1 |
| | cyclohexen-1-one | |

When present, each of the Moderately Compatible PRMs may be may be present at any level in the aqueous freshening composition. Preferably, each of the Moderately Compatible PRMs may be present in the aqueous freshening composition at a level up to 1.0 wt. %, or up to 0.6 wt. %, by weight of the aqueous freshening composition. Preferably the aqueous freshening composition comprises a total level of Moderately Compatible PRMs of 1.2 wt. % or less, by weight of the aqueous freshening composition.

Controlled PRMs

Perfume raw materials that have been found to provide minor crazing to pressurized plastic containers at low levels in the aqueous freshening composition may be selected from the group consisting of the perfume raw materials listed in Tables 3A and 3B and combinations thereof (“Controlled PRMs”).

TABLE 3A

| | IUPAC Name for | |
|---|---|---|
| Controlled PRMs | Controlled PRMs | CAS No |
| delta damascone | (z)-1-((1r,2s)-2,6,6- | 57378-68-4 |
| | trimethylcyclohex-3-en-1- | |
| | yl)but-2-en-1-one | |
| ionone beta | (e)-4-(2,6,6- | 14901-07-6 |
| | trimethylcyclohex-1-en-1- | |
| | yl)but-3-en-2-one | |
| floralozone | 3-(2-ethylphenyl)-2,2- | 67634-15-5 |
| | dimethylpropanal | |
| citronellol | 3,7-dimethyloct-6-en-1-ol | 106-22-9 |
| cymal | 3-(4-isopropylphenyl)-2- | 103-95-7 |
| | methylpropanal | |
| ionone alpha | (e)-4-(2,6,6- | 127-41-3 |
| | trimethylcyclohex-2-en-1- | |
| | yl)but-3-en-2-one | |
| undecalactone | 5-heptyldihydrofuran-2(3h)- | 104-67-6 |
| | one | |
| undecavertol | (e)-4-methyldec-3-en-5-ol | 81782-77-6 |
| 4-tertiary butyl cyclohexyl | 4-(tert-butyl)cyclohexyl | 32210-23-4 |
| acetate | acetate | |
| verdox | 2-(tert-butyl)cyclohexyl | 88-41-5 |
| | acetate | |
| allyl caproate | allyl hexanoate | 123-68-2 |
| ionone gamma methyl | (z)-3-methyl-4-(2,6,6- | 127-51-5 |
| | trimethylcyclohex-2-en-1- | |
| | yl)but-3-en-2-one | |
| dihydro myrcenol | 2,6-dimethyloct-7-en-2-ol | 18479-58-8 |
| dimethylbenzylcarbinylacetate | 2-methyl-1-phenylpropan- | 151-05-3 |
| | 2-yl acetate | |
| violiff | (z)-cyclooct-4-en-1-yl | 87731-18-8 |
| | methyl carbonate | |
| d-limonene | (r)-1-methyl-4-(prop-1-en- | 5989-27-5 |
| | 2-yl)cyclohex-1-ene | |
| frutene | 3a,4,5,6,7,7a-hexahydro- | 68912-13-0 |
| | 4,7-methano-1(3)h-inden-1- | |
| | yl propanoate | |
| linalool | 3,7-dimethylocta-1,6-dien- | 78-70-6 |
| | 3-ol | |
| linalyl acetate | 3,7-dimethylocta-1,6-dien- | 115-95-7 |
| | 3-yl acetate | |
| p.t. bucinal | 3-(4-(tert-butyl)phenyl)-2- | 80-54-6 |
| | methylpropanal | |
| cyclohexyl acetate | cyclohexyl acetate | 622-45-7 |
| p-cymene | p-cymene | 99-87-6 |
| ethyl linalool | (e)-3,7-dimethylnona-1,6- | 10339-55-6 |
| | dien-3-ol | |

TABLE 3B

| | IUPAC Name for | |
|---|---|---|
| Controlled PRMs | Controlled PRMs | CAS No |
| ionone (including gamma, | 1-(2,6,6-trimethyl-2- | |
| beta, alpha alpha-iso, etc.) | cyclohexen-1-yl)-1(3)- | |
| | penten-3(2)-one | |
| methyl-alpha-ionone | (e)-1-(2,6,6-trimethyl-1- | 127-42-4 |
| | cyclohex-2-enyl)pent-1-en- | |
| | 3-one (74-80%) | |
| florocyclene | 3a,4,5,6,7,7a-hexahydro- | 17511-60-3 |
| | 4,7-methano-1H-inden-6-yl | |
| | propionate | |
| floral acetate | 3a,4,5,6,7,7a-hexahydro- | 5413-60-5 |
| | 1H-4,7-methanoinden-6-yl | |
| | acetate | |
| cyclacet | 3a,4,5,6,7,7a-hexahydro- | 54830-99-8 |
| | 1H-4,7-methanoinden-1-yl | |
| | acetate | |
| 5-tricyclodecenyl acetate | 3a,4,5,6,7,7a-hexahydro- | 2500-83-6 |
| | 4,7-methano-1H-inden-5-yl | |
| | acetate | |
| methyl dioxolan | ethyl 2-(2-methyl-1,3- | 6413-10-1 |
| | dioxolan-2-yl)acetate | |
| lepidine | 4-methylquinoline | 491-35-0 |
| heptyl alcohol | 1-heptanol | 111-70-6 |
| Benzyl methyl ether | benzyl methyl ether | 538-86-3 |
| Dibenzyl ether | benzyl ether | 103-50-4 |
| p-Tolualdehyde | 4-methylbenzaldehyde | 104-87-0 |
| Isophorone | 3,5,5-trimethyl-2- | 78-59-1 |
| | cyclohexen-1-one | |

When present, each of the Controlled PRMs may be may be present in the aqueous freshening composition at a level up to 0.3 wt. %, more preferably up to 0.1 wt. %, by weight of the aqueous freshening composition. Preferably the aqueous freshening composition comprises a total level of Controlled PRMs of 0.6 wt. % or less, by weight of the aqueous freshening composition.

Limited PRMs

Perfume raw materials that have been found to provide minor crazing to pressurized plastic containers at low levels in the aqueous freshening composition may be selected from the group consisting of the perfume raw materials listed in Tables 4A and 4B and combinations thereof (“Limited PRMs”).

TABLE 4A

| | IUPAC Name for | |
|---|---|---|
| Limited PRMs | Limited PRMs | CAS No |
| ethyl-2-methyl butyrate | ethyl 2-methylbutanoate | 7452-79-1 |
| iso nonyl acetate | 3,5,5-trimethylhexyl | 58430-94-7 |
| | acetate | |
| manzanate (ethyl 2 methyl | ethyl 2-methylpentanoate | 39255-32-8 |
| pentanoate, ethyl 2 methyl | | |
| valerate) | | |
| sclareolate | propyl (s)-2-(tert- | 319002-92-1 |
| | pentyloxy)propanoate | |
| aphermate | 1-(3,3- | 25225-08-5 |
| | dimethylcyclohexyl)ethyl | |
| | formate | |

TABLE 4B

| | IUPAC Name for | |
|---|---|---|
| Limited PRMs | Limited PRMs | CAS No |
| lepidine | 4-methylquinoline | 491-35-0 |
| heptyl alcohol | 1-heptanol | 111-70-6 |
| floral acetate | 3a,4,5,6,7,7a-hexahydro- | 5413-60-5 |
| | 1H-4,7-methanoinden-6- | |
| | yl acetate | |
| cyclacet | 3a,4,5,6,7,7a- | 54830-99-8 |
| | hexahydro-1H-4,7- | |
| | methanoinden-1-yl | |
| | acetate | |
| 5-tricyclodecenyl acetate | 3a,4,5,6,7,7a-hexahydro- | 2500-83-6 |
| | 4,7-methano-1H-inden- | |
| | 5-yl acetate | |
| methyl dioxolan | ethyl 2-(2-methyl-1,3- | 6413-10-1 |
| | dioxolan-2-yl)acetate | |

When present, each of the Limited PRMs may be may be present in the aqueous freshening composition at a level up to 0.3 wt. %, more preferably up to 0.1 wt. %, more preferably up to 0.05 wt. %, more preferably up to 0.01 wt. %, by weight of the aqueous freshening composition. The aqueous freshening composition may be free of Limited PRMs. Preferably the aqueous freshening composition comprises a total level of Limited PRMs of 0.1 wt. % or less, by weight of the aqueous freshening composition.

The aqueous freshening composition may include one or more additional perfume raw materials. Exemplary perfume raw materials that may be present in the aqueous freshening composition includes the perfume raw materials listed in Table 5. The aqueous freshening composition may comprise a total perfume mixture level of about 0.001 wt. % to about 10 wt. %.

TABLE 5

| CAS No. | Name | Cas No. | Name |
|---|---|---|---|
| | Lime Aldehyde | 23696-85-7 | Damascenone |
| 1504-74-1 | Methoxycinnamaldehyde (Ortho) | 24048-13-3 | 2,6,10-Trimethyl-5,9-Undecadien- |
| 54082-68-7 | Onicidal (Muguet Undecadienal) | | 1-A1 |
| 1335-66-6 | Iso Cyclocitral | 24680-50-0 | Trans-4-Methoxycinnamaldehyde |
| 16630-52-7 | 3-Methylthiobutanal | 24851-98-7 | Methy-Dihydrojasmonate |
| | Formyl Tricyclodecan | 24851-98-7 | Hedione |
| Specialty | Corps Iris | 24851-98-7 | Methyl Dihydro Jasmonate |
| 120-14-9 | Corps 4322 (Vanillin Methyl | 25152-84-5 | 2,4-Decadienal |
| | Ether) | 2548-87-0 | Octenal |
| 93-08-3 | Methyl Beta Naphthyl Ketone | 2550-11-0 | Dimethyl-Octenone |
| | 6-Isopropyldecahydro-2-Naphtone | 2550-26-7 | Benzyl-Acetone |
| 123-69-3 | 8-Hexadecenolide | 26370-28-5 | 2,6-Nonadienal |
| | Eth-Me-Ph Glycidate Isomer | 27939-60-2 | Trivertal |
| 1335-46-2 | Xandralia (Methyl Ionone-Most | 29214-60-6 | Gelsone |
| | Common) | 30168-23-1 | Duplical |
| Specialty | Hs Raspberry | 30168-23-1 | Tricyclodecylidenebutanal |
| Specialty | Berry Wescorps | 30361-28-5 | 2,4-Octadienal |
| Specialty | Cassis Base | 30772-79-3 | Melozone |
| 11245-8 | Undec-10-En-1-Al (10-Undecenal) | 31375-17-4 | 1-(P-Menthen-6(2)-Yl)-1- |
| 41496-43-9 | 2-Methyl-3-Tolylproionaldehyde, | | Propanone |
| | 4-Dimethylbenzenepropanal (4- | 31906-04-4 | 4-(4-Hydroxy-4-Methyl Pentyl)-3- |
| | Dimethyl Benzenepropanal) | | Cyclohexene-1-Carboxaldehyde |
| | 4-Tricyclo5210-2,6decylidene- | 31906-04-4 | Cyclohexenyl-Carboxaldehyde |
| | 8butanal | 32210-23-4 | 4-Tertiary Butyl Cyclohexyl |
| 19009-56-4 | 2-Methyl Deca-1-Al (2 Methyl | | Acetate |
| | Decanal) | 32210-23-4 | Vertenex |
| 55418-52-5 | Cassione (Heliotropin Acetone) | 32388-55-9 | Methyl-Cedrenyl-Ketone |
| 100-06-1 | Para-Methoxy-Acetophenone | 32388-55-9 | Methyl Cedrylone Major |
| 10031-82-0 | 4-Ethoxybenzaldehyde | 32388-55-9 | Methyl-Cedrylone |
| 100-51-6 | Benzyl Alcohol | 33704-61-9 | Musk Indanone |
| 100-52-7 | Benzaldehyde | 33704-61-9 | 6,7-Dihydro-1,1,2,3,3- |
| 10094-34-5 | Dimethyl Benzyl Carbinyl Butyrate | | Pentamethyl-4(5h)-Indanone |
| 101-39-3 | Alpha-Methyl Cinnamic Aldehyde | 33885-51-7 | Pino Acetaldehyde |
| 101-39-3 | P-Methyl-Alpha- | 34590-94-8 | Dowanol DPM Isomer |
| | Pentylcinnamaldehyde | 34902-57-3 | Habanolide |
| 101-39-3 | 2-Methyl 3-Phenyl Propenal | 35044-59-8 | Ethyl Safranate |
| 101-39-3 | Methylcinnamaldehyde | 35044-68-9 | Beta-Damascone |
| 101-39-3 | Alpha-Methylcinnamaldehyde | 35044-68-9 | Damascone Beta |
| 101-48-4 | Phenyl Acetaldehyde Dimethyl | 35158-25-9 | 2-Isopropyl-5-Methyl-2-Hexenal |
| | Acetal | 36306-87-3 | 4-(1-Ethoxyvinyl)-3,3,5,5,- |
| 101-86-0 | Alpha-N-Hexyl-Cinnamaldehyde | | Tetramethyl-Cyclohexanone |
| 101-86-0 | 2-Hexyl 3-Phenyl Propenal | 3720-16-9 | Celery Ketone |
| 101-86-0 | Hexyl Cinnamic Aldehyde | 3720-16-9 | Livescone |
| 101-86-0 | Jasmonal H | 37609-25-9 | 5-Cyclohexadecenone |
| 101-86-0 | Alpha-Hexylcinnamaldehyde | 37677-14-8 | Myrac Aldehyde |
| 103-26-4 | Methyl Cinnamate | 39255-32-8 | Manzanate |
| 103-48-0 | Phenyl Ethyl Iso-Butyrate | 39255-32-8 | Ethyl 2 Methyl Pentanoate |
| 103-54-8 | Cinnamyl Acetate | 41496-43-9 | Jasmorange |
| 103-60-6 | Phenoxy Ethyl Iso-Butyrate | 41496-43-9 | Satinaldehyde |
| 103-95-7 | Alpha-Methyl-P-Isopropyl Phenyl | 41724-19-0 | Plicatone |
| | Propyl Aldehyde | 42370-07-0 | 2-Acetyl-3,3-Dimethyl-Norbornane |
| 103-95-7 | Cymal | 43052-87-5 | Damarose Alpha |
| 103-95-7 | Cyclosal | 43052-87-5 | Alpha-Damascone |
| 103-95-7 | Cyclamen Aldehyde | 432-25-7 | Beta-Cyclocitral |
| 103-95-7 | 2.Methyl-3(P-Isopropylphenyl)- | 4411-89-6 | 2-Phenyl 2-Butenal |
| | Propionaldehyde | 4411-89-6 | Phenyl Butenal |
| 103-95-7 | 3-(P-Isopropylphenyl)- | 4433-36-7 | 3,4,5,6-Tetrahydropseudoionone |
| | Propionaldehyde | 470-82-6 | Eucalyptol |
| 104-09-6 | Syringaldehyde | 472-66-2 | 2,6,6-Trimethyl-1-Cyclohexene-1- |
| 104-09-6 | P-Tolylacetaldehyde | | Acetaldehyde |
| 104-50-7 | Gamma-Octalactone | 472-66-2 | B-Homocyclocitral |
| 104-53-0 | Benzenepropanal | 4748-78-1 | 4-Ethyl Benzaldehyde |
| 104-55-2 | Cinnamic Aldehyde | 4819-67-4 | Delphone |
| 104-67-6 | Undecalactone | 488-10-8 | Cis-Jasmone |
| 10486-19-8 | Tridecanal | 491-35-0 | Lepidine |
| 105-95-3 | Ethylene Brassylate | 4927-36-0 | 4-Damascol |
| 106-02-5 | Pentadecanolide | 4940-11-8 | Ethyl Maltol |
| 106-22-9 | Citronellol | 495-85-2 | Amylaldehyde |
| 106-23-0 | 3,7-Dimethyl 6-Octenal | 502-72-7 | Cyclopentadecanone |
| 106-23-0 | Citronellal | 51414-25-6 | Lyral |
| 106-24-1 | 3,7-Dimethyl-2,6-Octadien-1-Al | 52474-60-9 | Precyclemeone B |
| 106-26-3 | Neral | 5392-40-5 | Citral |
| 106-72-9 | 2,6-Dimethyl-5-Heptenal | 5392-40-5 | Geranial |
| 106-72-9 | Melonal | 541-91-3 | Muscone |
| 107-75-5 | 3,7-Dimethyl Octan-1-Al | 54464-57-2 | Iso-E-Super |
| 107-75-5 | Hydroxycitronellal | 54464-57-2 | Isocyclemone E |
| 107-75-5 | Citronellal Hydrate | 5462-06-6 | Canthoxal |
| 107-75-5 | 7-Hydroxy-3,7-Dimethyl Octan-1- | 5462-06-6 | Anisylpropanal |
| | Al | 5471-51-2 | Para Hydroxy Phenyl Butanone |
| 107-86-8 | 3-Methyl-2-Butenal | 55066-49-4 | 3-Methyl-5-Phenyl Pentanal |
| 107898-54-4 | Polysantol | 55066-49-4 | Mefranal |
| 108-29-2 | Gamma-Valero Lactone | 55418-52-5 | Dulcinyl |
| 110-41-8 | 2-Methyl-1-Undecanal | 564-94-3 | Myrtenal |
| 110-41-8 | Methyl Nonyl Acetaldehyde | 564-94-3 | Pin-2-Ene-1-Carbaldehyde |
| 110-41-8 | Aldehyde C12 MNA | 56973-85-4 | Neobutenone |
| 110-62-3 | Pentanal | 5703-26-4 | 4-Methylphenylacetaldehyde |
| 110-62-3 | Valeraldehyde | 57378-68-4 | Delta-Damascone |
| 110-93-0 | Methyl-Heptenone | 57934-97-1 | Givescone |
| 111-30-8 | Glutaraldehyde | 58430-94-7 | Iso-Nonyl Acetate |
| 111-30-8 | Pentanedial | 590-86-3 | 3-Methyl Butyraldehyde |
| 111-30-8 | Glutaric Aldehyde | 590-86-3 | Isovaleraldehyde |
| 111-71-7 | Heptanal | 59323-76-1 | Oxane |
| 112-12-9 | Methyl Nonyl Ketone | 5988-91-0 | Dihydrocitronellal |
| 112-31-2 | Decanal | 5989-27-5 | D-Limonene |
| 112-44-7 | Undecenal | 60-12-8 | Phenyl Ethyl Alcohol |
| 112-54-9 | Lauric Aldehyde | 613-69-4 | 2-Ethoxybenzaldehyde |
| 112-54-9 | 2-Dodecanal | 621-59-0 | 4-Methoxy 3-Hydroxy |
| 1128-08-1 | Dihydrojasmone | | Benzaldehyde |
| 115-95-7 | Linalyl Acetate | 623-36-9 | 2-Methyl-2-Pentenal |
| 116-26-7 | 2,6,6-Trimethyl-1,3-Diene | 623-36-9 | 2-Methylpentenal |
| | Methanal | 62439-41-2 | Methoxy Melonal |
| 116-26-7 | Safranal | 62439-41-2 | 6-Methoxy-2,6-Dimethylheptanal |
| 118-58-1 | Benzyl Salicylate | 62518-65-4 | Mefloral |
| 1191-16-8 | Prenyl Acetate | 62518-65-4 | Lilestralis 33 |
| 1192-88-7 | 1-Cyclohexene-1-Carboxaldehyde | 628-63-7 | Amyl-Acetate |
| 119-36-8 | Methyl Salicylate | 6413-10-1 | Fructone |
| 1195-79-5 | Fenchone | 65443-14-3 | Veloutone |
| 119-61-9 | Benzophenone | 65885-41-8 | Beta Methyl Benzenepropanal |
| 120-14-9 | 3,4-Dimethoxybenzaldehyde | 659-70-1 | Iso-Amyl Iso-Valerate |
| 120-14-9 | Veratraldehyde | 66-25-1 | Hexenal |
| 120-51-4 | Benzyl Benzoate | 66327-54-6 | 1-Methyl-4-(4-Methylpentyl)-3- |
| 1205-17-0 | 2-Methyl-3-(3,4- | | Cyclohexenecarbaldehyde |
| | Methylenedioxyphenyl)Propanal | 66327-54-6 | Vemaldehyde |
| 1205-17-0 | Helional | 6728-26-3 | 2-Hexenal |
| 120-57-0 | 3,4-Methylene Dioxy | 6728-31-0 | Cis Heptenal |
| | Benzaldehyde | 6753-98-6 | Alpha-Caryophyllene |
| 120-57-0 | Heliotropin | 67633-95-8 | Methyl-Lavender-Ketone |
| 120-72-9 | Indole | 67634-14-4 | Para-Ethyl-Alpha, Alpha-Dimethyl |
| 121-32-4 | 3-Ethoxy 4-Hydroxybenzaldehyde | | Hydrocinnamaldehyde |
| 121-32-4 | Ethyl Vanillin | 67634-14-4 | Floralozone |
| 121-33-5 | Vanillin | 67801-65-4 | Triplal Extra |
| 122-00-9 | Methyl-Acetophenone | 67845-30-1 | Maceal |
| 122-03-2 | 4-Isopropyl Benzaldehyde | 68039-49-6 | 2,4-Dimethyl-3-Cyclohexene-1- |
| 122-03-2 | Cuminaldehyde | | Carboxaldehyde |
| 122-40-7 | Amyl Cinnamic Aldehyde | 68039-49-6 | Cyclal C |
| 122-40-7 | Alpha-Amylcinnamic Aldehyde | 68039-49-6 | Ligustral |
| 122-40-7 | 2-Pentyl-3-Phenylpropenoic | 68039-49-6 | Triplal |
| | Aldehyde | 99-49-0 | Carvone |
| 122-48-5 | 4-(4-Hydroxy-3-Methoxyphenyl)- | 68039-49-6 | Vertocitral |
| | 2-Butanone | 68039-49-6 | 2,4-Dimethyl-3-Cyclohexen-1- |
| 122-78-1 | Phenylacetaldehyde | | Carbaldehyde |
| 122-97-4 | Phenyl Propyl Alcohol | 68480-14-8 | Methyl Cyclocitrone |
| 123-11-5 | P-Methoxybenzene Aldehyde | 68737-61-1 | 2,4-Dimethylcyclohex-3-Ene-1- |
| 123-11-5 | Anisic Aldehyde | | Carbaldehyde |
| 123-11-5 | Anisaldehyde | 68738-96-5 | Cyclemone A |
| 123-15-9 | 2-Methyl Valeraldehyde | 68912-13-0 | Frutene |
| 123-15-9 | 2-Methylpentanal | 68991-97-9 | Melafleur |
| 123-38-6 | Propanal | 68991-97-9 | 1,2,3,4,5,6,7,8-Octahydro-8,8- |
| 123-38-6 | Propionaldehyde | | Dimethyl-2-Naphthaldehyde |
| 123-68-2 | Allyl Caproate | 70266-48-7 | Iso-Damascone |
| 123-72-8 | Butyraldehyde | 706-14-9 | Gamma Decalactone |
| 124-13-0 | Octanal | 71077-31-1 | Floral Super |
| 124-19-6 | Nonanal | 74338-72-0 | 2,4,4,7-Tetramethyl-Oct-6-En-3- |
| 125109-85-5 | 3-(3-Isopropyl-Phenyl)- | | One |
| | Butyraldehyde | 7452-79-1 | Ethyl-2-Methyl Butyrate |
| 125109-85-5 | Florhydral | 74568-05-1 | Gamma Undecalactone |
| 127-41-3 | Alpha-Ionone | 7492-67-3 | 3,7-Dimethyl-6-Octenyl |
| 127-42-4 | Alpha-Methyl Ionone | | Oxyacetaldehyde |
| 127-43-5 | N-Beta-Methyl Ionone Isomer | 7492-67-3 | Citronellyl Oxyacetaldehyde |
| 127-51-5 | Gamma-Methyl Ionone | 7492-67-3 | Muget Aldehyde 50 |
| 128-37-0 | BHT | 75-07-0 | Ethanal |
| 1322-58-3 | Tetrameran | 75-07-0 | Acetaldehyde |
| 1335-66-6 | Iso-Cyclo Citral | 75147-23-8 | Buccoxime |
| 1335-66-6 | 2,4,6-Trimethyl-3-Cyclohexene-1- | 76-22-2 | Camphor |
| | Carboxaldehyde | 7775-00-0 | Cyclemax |
| 1335-66-6 | Iso Cyclocitral | 78-70-6 | Linalool |
| 1337-83-3 | Intreleven Aldehyde | 78-84-2 | Isobutyraldehyde |
| 134-96-3 | 3,5-Dimethoxy 4- | 78-98-8 | Pyruvaldehyde |
| | Hydroxybenzaldehyde | 79-76-5 | Gamma-Ionone |
| 137-03-1 | Fleuramone | 79-78-7 | Hexalon |
| 139-85-5 | 3,4-Dihydroxybenzaldehyde | 8028-48-6 | Orange Oil Tarocco |
| 139-85-5 | Catechaldehyde | 80-54-6 | 2-Methyl-4-T- |
| 140-11-4 | Benzyl Acetate | | Butylphenyl)Propanal |
| 141-13-9 | 2,6,10-Trimethyl-9-Undecenal | 80-54-6 | 4-Tert-Butyl-Alpha-Methyl- |
| 141-13-9 | Adoxal | | Hydrocinnamaldehyde |
| 141773-73-1 | Helvetolide | 80-54-6 | Lilial |
| 142-83-6 | 2,4-Hexadienal | 80-54-6 | P.T. Bucinal |
| 14371-10-9 | Phenyl Propenal, 3-Phenyl-2- | 80-54-6 | Lysmeral |
| | Propenal | 98-53-3 | Para-Tert-Butyl-Cyclohexanone |
| 14765-30-1 | Freskomenthe | 80-56-8 | Alpha-Pinene |
| 14901-07-6 | Beta-Ionone | 81782-77-6 | Undecavertol |
| 14901-07-6 | Ionone Beta | 82461-14-1 | Rhubafuran |
| 151-05-3 | Dimethyl Benzyl Carbinyl Acetate | 84697-09-6 | Acalea |
| 15764-16-6 | 2,4-Dimethylbenzaldehyde | 85-91-6 | Dimethyl Anthranilate |
| 16251-77-7 | Trifernal | 86803-90-9 | Scentenal |
| 16251-77-7 | 3-Phenyl Butanal | 86803-90-9 | Octahydro-5-Methoxy-4,7- |
| 16587-71-6 | Orivone | | Methano-1H-Indene-2- |
| 17283-81-7 | Dihydro-Beta-Ionone | | Carboxaldehyde |
| 1728-46-7 | Verdone | 88-41-5 | Verdox Major |
| 173445-65-3 | Neo Hivernal | 88-41-5 | Verdox |
| 18127-01-0 | 4-T-Butylbenzenepropionaldehyde | 89-80-5 | Menthone |
| 18127-01-0 | Bourgeonal | 90-02-8 | 2-Hydroxy Benzaldehyde |
| 18479-58-8 | Dihydro Myrcenol | 90-02-8 | Salicylaldehyde |
| 18829-55-5 | Trans Heptenal | 90105-92-3 | Prunella |
| 18829-56-6 | Nonenal | 90-87-9 | Hydrotropaldehyde |
| 19009-56-4 | Methyl Octyl Acetylaldehyde | 91462-24-7 | Cyclic Ethylene Dodecanedioate |
| 19009-56-4 | Aldehyde C-11 MOA | 91-64-5 | Coumarin |
| 20407-84-5 | Mandarine Aldehyde | 928-96-1 | Beta-Gamma Hexenol |
| 20407-84-5 | Mandarinal | 93-08-3 | Methyl-Beta-Naphthyl-Ketone |
| 20665-85-4 | Vanillin Isobutyrate | 93-16-3 | Methyl Isoeugenol |
| 2111-75-3 | L-4(1-Methylethenyl)-1- | 93-28-7 | Eugenyl Acetate |
| | Cyclohexene-1-Carboxaldehyde | 93-53-8 | 2-Phenylproprionaldehyde |
| 2111-75-3 | Perillaldehyde | 93-92-5 | Methyl Phenyl Carbinyl Acetate |
| 21145-77-7 | Tonalid | 95-41-0 | Iso Jasmone |
| 21145-77-7 | Musk Plus | 95962-14-4 | 2-(2-(4-Methyl-3-Cyclohexen-1- |
| 21944-98-9 | Tangerinal | | Yl)Propyl)-Cyclopentanone |
| 22471-55-2 | Thesaron | 96-17-3 | 2-Methyl Butyraldehyde |
| 2349-07-7 | Hexyl Iso-Butyrate | 96-17-3 | Methylbutyraldehyde |
| 23696-85-7 | Damascenone | 97-53-0 | Eugenol |
| 98-86-2 | Acetophenone | 97-96-1 | 2-Ethylbutyraldehyde |

The aqueous freshening composition may be substantially free of or free of a flavorant. A flavorant is an edible chemical that is added to food and beverage products to alter the taste of the food or beverage product. Where a perfume mixture is free of a flavorant, the perfume mixture is free of flavorants including perfume raw materials that are known to be used as flavorants in the food and beverage industry. Having a perfume mixture that is free of flavorants can help provide improved hedonic benefits of the aqueous freshening composition.

Aqueous Carrier

The aqueous freshening composition also comprises an aqueous carrier. The aqueous carrier which is used may be distilled, deionized, or tap water. Water may be present in any amount for the composition to be aqueous. In some embodiments, water may be present in an amount of about 70% to about 99.9%, or about 80% to about 99.9%, or about 85% to 99.9%, or about 90% to about 99.5%, or about 92% to about 99.5%, or about 95%, by weight of said aqueous freshening composition.

Additional Ingredients

The aqueous freshening composition may also have less than about 30%, or less than about 10%, or less than about 5%, by weight of the composition, of alcohol. However, the volatile low molecular weight monohydric alcohols such as ethanol and/or isopropanol should be limited since these volatile organic compounds may contribute both to flammability problems and environmental pollution problems. If small amounts of low molecular weight monohydric alcohols (e.g., ethanol, methanol, and isopropanol, or polyols, such as ethylene glycol and propylene glycol) may be in present in the composition due to the addition of these alcohols to such things as perfumes and as stabilizers for some preservatives, the level of monohydric alcohol may be about 1% to about 5%, alternatively less than about 5%, by weight of the aqueous freshening composition.

The aqueous freshening composition of the present disclosure may be formulated into an aerosol freshening composition comprising malodor counteractants, particulate controlling polymers, emulsifiers and solubilizing surfactants to solubilize any excess hydrophobic organic materials, particularly any perfume raw materials. Other emulsifiers, solvents, solubilizers and surfactants as described in U.S. Pat. No. 7,998,403 or in US 2012/0288448A1 can be used to enhance performance of the aqueous freshening composition. A suitable solubilizing surfactant, is a no-foaming or low-foaming surfactant. In one embodiment, the aqueous freshening composition contains ethoxylated hydrogenated castor oil. One suitable hydrogenated castor oil is Basophor™, available from BASF. The solubilizer to perfume ratio in aqueous freshening composition may be about 2:1 or greater than 2:1.

Pressurized Plastic Container

The aqueous freshening compositions of the present disclosure have been found to be compatible with and useful in pressurized plastic containers. “Plastic” refers to any synthetic or organic material that can be molded or shaped, generally when heated, and then hardened into a desired form including, but not limited to, polymer, resin, and cellulose derivative. The plastic may be polymeric and may be partially, substantially, or entirely comprised of polyester; polyethyleneterephthalate (“PET”); polyethylene napthalate, polyethylene furanoate, polyamide; nylon 6/6, nylon 66, nylon 11, polycarbonate; polyoxymethylene; polyacrylonitrile; polyolefin; polyethylene, polypropylene, fluoropolymer; poly(butylene succinate); virgin, recycled, and regrind versions of the other polymer materials; bio-based and petroleum-based versions of the other polymer materials; and mixtures thereof. The pressurized plastic container may comprise multiple layers of other polymer materials. By polymeric it is meant that the component is formed of a material which is plastic, comprises polymers, and/or particularly polyolefin, polyester or nylons. Thus, the entire pressurized plastic container or, specific components thereof, may be free of metal, allowing for exposure to microwave energy.

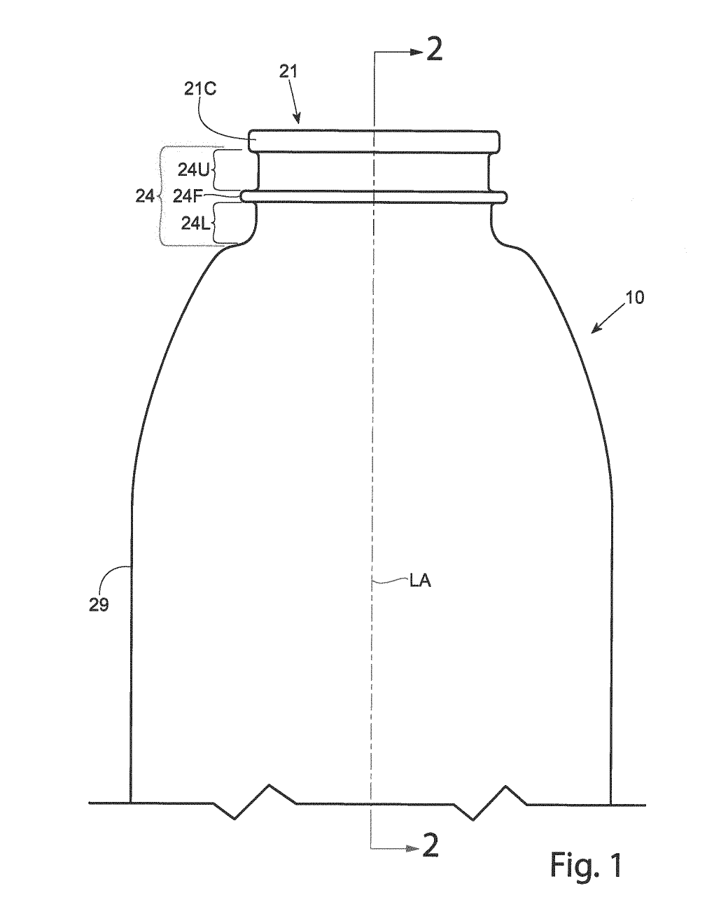

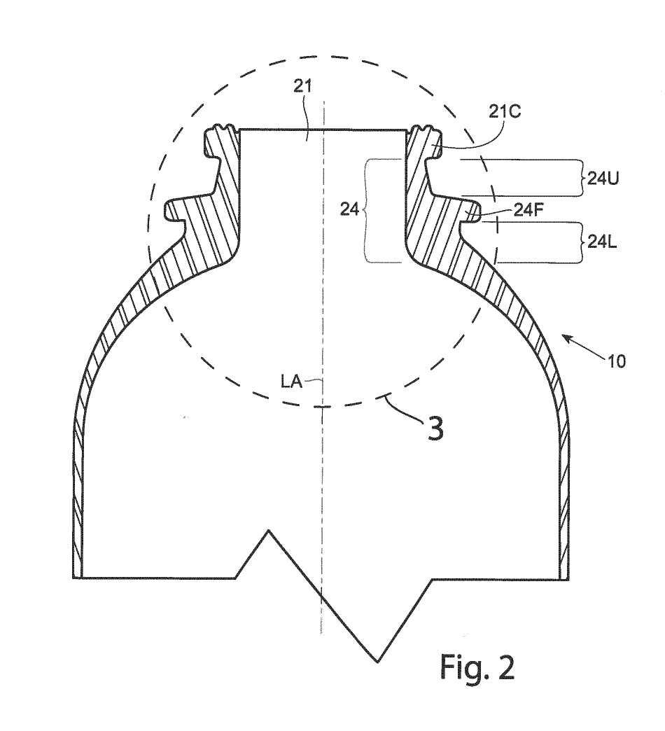

Referring FIGS. 1 and 2, as the top of plastic container 10 is approached, the plastic container may have an opening 21. An optional crimp ring 21C may be circumjacent, and in a degenerate case circumscribe the opening 21. The optional crimp ring 21C may provide for attachment of an optional valve cup as is known in the art.

Below the optional crimp ring 21C, is a neck 24. The neck 24 may be of constant or variable cross section. The neck 24 may have an optional flange 24F. The optional flange 24F may extend radially outward from the neck 24, as shown and/or may extend radially inwardly. The flange 24F may circumscribe the neck 24 or may be interrupted at various positions around the circumference to be discontinuous. The plastic container 10 may have a step in the outer wall of the neck 24. Such a plastic container may be made according to U.S. Pat. No. 6,971,530. The flange 24F may be disposed near the axial center of the neck 24, as shown, or may be juxtaposed with the top or bottom of the neck 24. The flange 24F may divide the neck into an upper portion 24U and a lower portion 24L. The neck 24 may have a lesser thickness at the top portion 24U than at lower portion 24L, or vice versa, to provide a differential thickness.

The lower portion 24L of the neck 24 may be above and/or superjacent the shoulder 25 of the plastic container 10. The shoulder 25 may flare radially outwardly from the lower portion 24L in a first transition region. The shoulder 25 may connect to the container sidewall 29. The shoulder 25 may particularly be joined to the sidewall 29 by a radius or second transition region. The plastic container 10 sidewall also defines a diameter if a round cross section is selected for the body.

The plastic container 10 is pressurized to an internal gage pressure of about 345 kPa (55 psi) to about 1100 kPa, or from about 414 kPa to about 1100 kPa, or from about 460 kPa to about 1100 kPa, or from about 480 kPa to about 1100 kPa, or from about 500 to about 1100 kPa, or from about 550 kPa to about 1100 kPa, or from about 600 kPa to about 1100 kPa, or from about 690 kPa (100 psi) to about 1100 kPa, or about 827 kPa (120 psi) to about 1100 kPa, or 935 kPa (135 psi) to about 1100 kPa.

A plastic container 10 having a crystallized neck may be pressurized to an internal gage pressure of up to 1300 kPa, preferably 345 kPa to 1300 kPa, or 414 kPa to about 1100 kPa, or from about 460 kPa to about 1100 kPa, or from about 480 kPa to about 1100 kPa, or from about 500 to about 1100 kPa, or from about 550 kPa to about 1100 kPa, or from about 600 kPa to about 1100 kPa, or from about 690 kPa (100 psi) to about 1100 kPa, or about 827 kPa (120 psi) to about 1100 kPa, or 935 kPa (135 psi) to about 1100 kPa or preferably from about 460 kPa to about 1100 kPa.

The final gage pressure of the plastic container is 0 when the propellant is fully discharged from the container. Where residual aqueous freshening composition remains, the final gage pressure may be from about 0 to about 120 kPa.

The pressurized plastic container 10 includes a propellant. Any suitable propellant may be used. The propellant may comprise a compressed gas such as, nitrogen, carbon dioxide, compressed air, and mixtures thereof. The propellant may comprise a liquefied gas or hydrofluoro olefin (“HFO”) propellant. If a liquefied gas propellant is used, the pressurized plastic container may include a bag-in-bottle configuration. Propellants listed in the U.S. Federal Register 49 C.F.R. §1.73.115, Class 2, Division 2.2 are considered acceptable. The propellant may particularly comprise a trans-1,3,3,3-tetrafluoroprop-1-ene, and optionally a CAS number 1645-83-6 gas. Such propellants provide the benefit that they are not flammable, although the composition is not limited to inflammable propellants. One such propellant is commercially available from Honeywell International of Morristown, N.J. under the trade name HFO-1234ze or GWP-6.

If desired, the propellant may be condensable. By “condensable”, it is meant that the propellant transforms from a gaseous state of matter to a liquid state of matter in the container and under the pressures encountered in use. Generally, the highest pressure occurs after the container is charged with product but before that first dispensing of that product by the user. A condensable propellant provides the benefit of a flatter depressurization curve as product is depleted during usage.

The volumetric ratio of aqueous freshening composition to propellant may be in the range of about 40/60 to about 70/30, alternatively in the range of about 50/50 to about 60/40.

The aqueous freshening composition is delivered from the pressurized plastic container 10 which includes delivery components including but not limited to a valve to control flow and to seal the composition within the pressurized plastic container, a button actuator and a nozzle for dispensing the composition to the environment.

The aqueous freshening composition may contact the inner face of the plastic container 10 and is not contained in a plastic container by a bag-in-can or a plastic container comprising a liner.

In other embodiments, the aqueous freshening composition may be contained in a bag-in-can or bag-in-bottle plastic container.

The pressurized plastic container may hold from about 20 grams to about 300 grams, or at least about 20 grams, or at least about 120 grams, or at least about 130 grams, or at least about 150 grams.

The container comprises a hoop tensile strain from about 0.1% to about 2%, or from about 0.1% to about 1%, or from about 0.2% to about 0.6%. “Hoop tensile strain” is the resultant material deformation as a function of the internally applied force exerted circumferentially (perpendicular to the major longitudinal axis LA on every particle in the cylinder wall of a plastic container and is represented as a % value derived from the hoop tensile strain test method set forth below.

Crazing of about 1 mm or greater through a plastic PET is typically visible to the human eye and may compromise product aesthetics. It may compromise the function of the container under use conditions (e.g. may result in container becoming more brittle or rupturing when force is applied to it, for example, container dropping). Typically, the deeper the craze through the plastic material, the more compromised the integrity of the plastic becomes.

Preferably, the freshening product is stable in the pressurized plastic container for 1 year, more preferably 2 years, more preferably 3 years, most preferably 5 years.

The total composition output and the spray droplet/particle size distribution may be selected to support the particulate removal efficacy but avoid a surface wetness problem. Total output is determined by the flow rate of the composition it is released from the spray dispenser. To achieve a spray profile that produces minimal surface wetness, it is desirable to have a low flow rate and small spray droplets.

Flow rate is determined by measuring the rate of composition expelled by a container for any 60 seconds period of use. The flow rate of the composition being released from the spray dispenser may be from about 0.0001 grams/second (g/s) to about 2.5 grams/second. Alternatively, the flow rate may be from about 0.001 grams/second to about 1.8 grams/second, or about 0.01 grams/second to about 1.6 grams/second.

The mean particle size of the spray droplets may be in the range of from about 10 μm to about 100 μm, alternatively from about 20 μm to about 60 μm. At least some of the spray droplets are sufficiently small in size to be suspended in the air for at least about 10 minutes, and in some cases, for at least about 15 minutes, or at least about 30 minutes.

Small particles can be efficiently created when the spray is dispensed in a wide cone angle. For a given nozzle component and delivery tube, cone angles can be modified by varying the insertion depth of the nozzle in the delivery tube. The cone angle may be greater than about 20 degrees, or greater than about 30 degrees, or greater than about 35 degrees, or greater than about 40 degrees, or greater than about 50 degrees.

The spray dispenser may be configured to spray the composition at an angle that is between an angle that is parallel to the base of the container and an angle that is perpendicular thereto. The desired size of spray droplets can be delivered by other types of spray dispensers that are capable of being set to provide a narrow range of droplet size. Such other spray dispensers include, but are not limited to: foggers, ultrasonic nebulizers, electrostatic sprayers, and spinning disk sprayers.

The aqueous freshening composition may also be formulated for use in personal care products such as skin moisturizers, body deodorants, facial and body cleansers, baby wipes; surface care compositions such as hard surface cleaners, wood polishes, and automobile cleaners; fabric care compositions such as cleaners, softeners, de-wrinklers, and refreshers; and air freshening compositions including aerosols and sprays.

The freshening products may be used to freshen the air, surfaces, fabrics, and/or combinations thereof.

Test Method to Determine % Hoop Tensile Strain at Neck—Measurement of Pressurized Plastic Aerosol Container Strain

Optical (photographic 2D), X-ray (Computed Tomography 3D), or equivalent imaging techniques may be used to quantify the level of strain at specific locations on a pressurized aerosol container. The % hoop tensile strain determination is made by imaging the region of interest with and without pressure. Relative dimensional changes are used to calculate % hoop tensile strain.

Sample Preparation

Procure a minimum of six unused pressurized plastic aerosol containers and condition them for 24 hours in the ambient environment (24° C.+/−3° C.) prior to imaging.

Imaging

Secure each conditioned plastic container by the upper neck 24U in a collet or equivalent clamping device to keep the container stable and to prevent contact with surrounding surfaces. Orient the containers, relative to the imaging device, such that at least three containers will be imaged, displaying their widest diameter of the neck 24 and at least three other containers will be imaged displaying their smallest diameter of the neck. The container temperature, when imaged, should be held constant across the pressurized and unpressurized conditions.

For each sample, optimize the resolution by only imaging the region of interest—the lower neck 24L and filling the field of view. Fix the imaging device field of view/focal plane. Place a scale that occupies the field of view in the focal plane and capture the image to set the image scale. The minimum resolution is 100 pixels/mm or 64,000 voxels/mm³. Place the plastic container in the field of view/focal plane and capture the image using appropriate imaging techniques to maximize contrast between the plastic container and the environment. Relieve the aerosol pressure without moving the plastic container or altering the position of the field of view or focal plane. Allow the unpressurized plastic container to equilibrate for a minimum of 1 hour so that the external pressure is equal to the internal pressure, while ensuring that there is no movement of the plastic container, the field of view, or the focal plane. Capture the unpressurized plastic container image under the same imaging conditions as the pressurized plastic container.

Image Analysis:

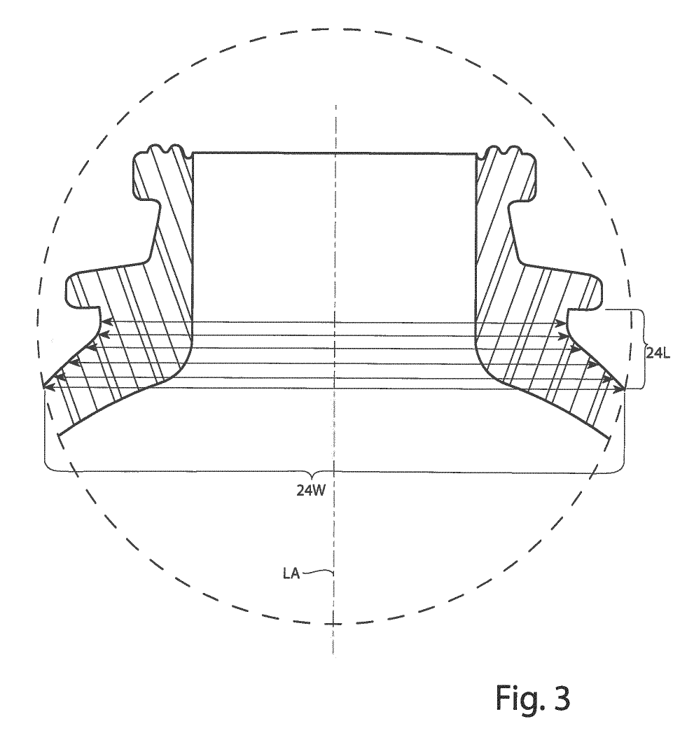

Using appropriate image analysis software, such as Image J (National Institutes of Health, Bethesda, Md., U.S.A.), or Geomagic Studio (Geomagic Inc., Morrisville, N.C., U.S.A.), set the dimensional scale in the images (pixels/mm or voxels/mm³) by using the previously captured scale image. Measure the outside diameter (“OD”) of the pressurized lower neck 24L image at 1 mm increments, defined as starting 1 mm below the flange 24F or below the neck 24 MidPoint MP and extending 5 mm vertically toward the bottom of the container 10, generating a total of 6 OD measurements as denoted with lines 24W shown in FIG. 3. Precisely identify the six locations where outside diameters were measured in the pressurized neck image, then remeasure the outside diameter at these same six locations in the unpressurized neck image. Convert the linear outside diameter dimensional change to % hoop tensile strain per the following equation, and report the maximum % hoop tensile strain value from the six locations measured on each container:

% Hoop Tensile Strain at location x=(ODPₓ−ODUₓ)/ODPₓ*100

where ODPₓ=Outside diameter pressurized at location x, where x=1 to 6; where ODUₓ=Outside diameter unpressurized at location x, where x=1 to 6; and where the strain value recorded for each container is the maximum value obtained from the six locations measured. The % Hoop Tensile Strain value reported is the maximum strain value recorded from the six (or more) individual containers imaged.

Plastic Container Lower Neck 24L Hoop Strain Determination Example:

Imaging

Image lower neck 24L, capturing the full width 24W while setting the focal plane at the maximum neck diameter. Image pressurized and post pressurization region as instructed in the imaging section above.

| | Post pressure | | | | | | | Strain | Strain | Strain | Strain | Strain | Strain | Max |
|---|---|---|---|---|---|---|---|---|---|---|---|---|---|---|
| Identification | time (minutes) | OD 1 | OD 2 | OD 3 | OD 4 | OD 5 | OD 6 | 1 (%) | 2 (%) | 3 (%) | 4 (%) | 5 (%) | 6 (%) | Strain (%) |
| Pressurized | | 23.86 | 21.16 | 20.48 | 20.14 | 19.84 | 19.73 | | | | | | | 0.30 |
| Unpressurized | 150 | 23.86 | 21.11 | 20.43 | 20.08 | 19.82 | 19.70 | 0.00 | 0.24 | 0.24 | 0.30 | 0.10 | 0.15 | |

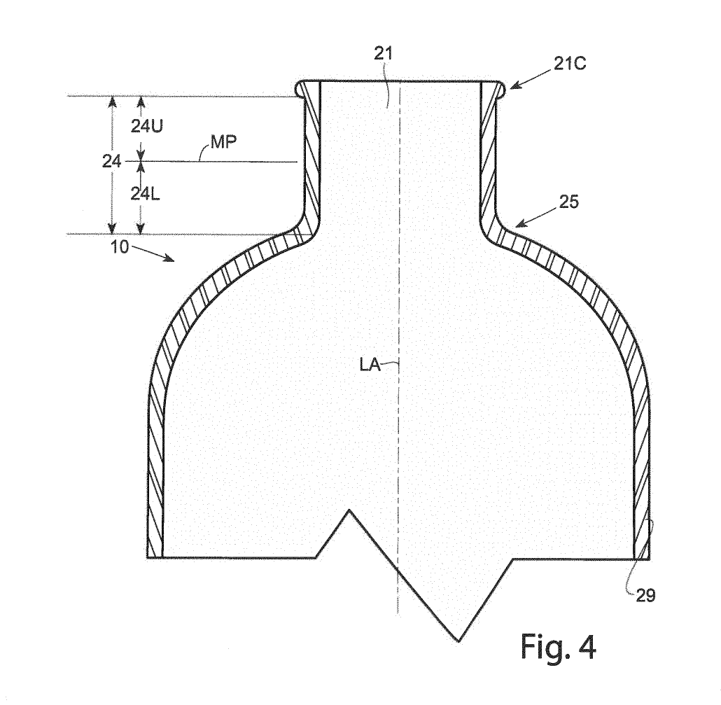

Referring to FIG. 4, the plastic container 10 may not have the optional flange 24F. In such case, the hoop tensile strain may be taken as a region below the axial midpoint MP of the neck 24. In determining the axial midpoint MP of the neck 24, only the portion of the neck 24 having constant cross section, if present, is considered. If the neck 24 has a variable cross section, the axial midpoint MP is that point of the neck 24 intermediate and midway between the underside of the optional crimp ring 21C and the start of the shoulder 25. If the plastic container 10 does not have a crimp ring 21C, the top of the neck 24 is utilized instead. The axial midpoint MP of the neck 24 may be used in addition to or in place of the flange 24F for purposes described and claimed herein.

As measured maximum strain levels in amorphous bottle neck regions can be replicated utilizing amorphous PET test bars. Standard test bars, about bottle neck thickness, are injection molded utilizing bottle PET under conditions replicating preform molding. Inverted bottle neck (internal formula contact) craze formation has been found to correlate with immersed test bar results. Strained test bars can be used to quickly quantify crazing potential of formulations in direct contact with amorphous PET.

Test Method to Determine Crazing Effect of Perfume Raw Materials and Plastic Material Under Strain

To simulate hoop tensile strain of a pressurized plastic container, injection molded PET test bars of about 127 mm long×12.7 mm wide×3.1 mm thick were clamped to individual curved stainless steel fixtures, putting the outer surface of bars under bending strain per equation:

ε=h/(d+h)

Where

h=thickness of test bars (eg. mm, m, in)

d=diameter of fixture (eg. mm, m, in)

ε=unit less measure of strain

ε%=*100

ISO 22088-3/2006 describes details for determining strain test parameters. The test bars were molded to intrinsic viscosity of about 0.79±0.02 dl/g per ASTM D4603, with an average maximum molded IV drop of 0.06 dl/g vs starting PET resin pellets.

Test bars of similar age range were used for these comparative studies as PET properties can change over time with storage conditions (temperature, humidity). Test bars used for these experiments were stored at room temperature (about 24±3 C) for a maximum of one month after initial molding; otherwise, test bars were annealed to about 10° C. above glass transition temperature for about 15 minutes and cooled slowly on a flat inert surface to prevent warping. Post annealing or injection molding, all bars are conditioned/equilibrated at room temperature for minimum of two hours before clamping on test fixtures.

Formulation samples were prepared as discussed below with regard to Tables 6 and 8. The formulation ingredients were mixed together to create clear and single phase solutions. The PRM, surfactants, emulsifiers and solvents were premixed together as needed to emulsify the PRM into solution. All surfactants, emulsifiers, buffers, stabilizers in Tables 6 and 8 are identical and were previously tested to ensure no additional impact on test bars.

The whole fixtures were totally immersed in sealed glass containers with formulations indicated in Tables 6, 7, 8, and 9 and then stored at room temperature for 24 hours. The bars were then removed from the glass containers, rinsed with DI water, and allowed to dry. The test bars were then placed under a microscope and oriented relative to the imaging device and LED light source, such that the full thickness in the middle of the bar is in field of view. Images were then captured using calibrated imaging scale, with a minimum of 100 pixels/mm The depth of the deepest craze observed on each test bar was measured using appropriate image analysis software, such as DinoCapture (Energiestraat, Naarden, The Netherlands), Image J (National Institutes of Health, Bethesda, Md., U.S.A.), or Geomagic Studio (Geomagic Inc., Morrisville, N.C., U.S.A.).

% Crazed is then calculated by:

(Deepest Craze Depth(mm)/Total Thickness of Bar(mm)*100

The higher the % Crazed, the less compatible the formulation is with the pressurized plastic container; conversely, the lower the % Crazed, the more compatible and less interactive the formulation is with the pressurized plastic container

At least 3 test bars were evaluated per formulation and then % Crazed is averaged per formulation tested.

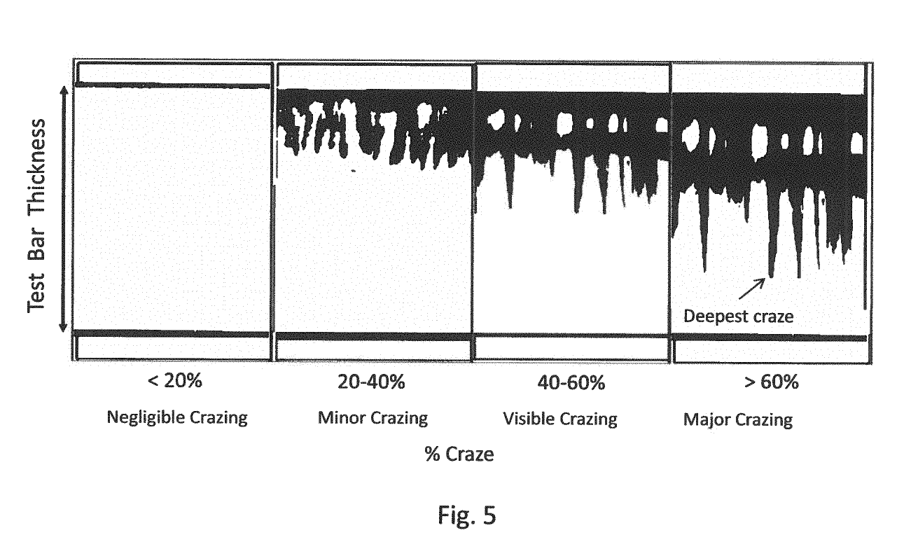

FIG. 5 illustrates visual examples of crazing observed at different % Crazed ranges. Crazing of 20% or less is virtually negligible, crazing below 40% is considered minor and may not be visible to the typical human eye. Crazing above 40% is typically visible and may be consumer unacceptable aesthetically. Crazing above 60% is very visible to the human eye and may also compromise the function of the container under use conditions (e.g. may result in container rupturing when force is applied to it, for example, container dropping).

Perfume Raw Material Interaction with Plastic Material Under Strain

Formulations were prepared to test the effect of individual PRMs on plastic with and without strain. Formulations were prepared according to Table 6, with each formulation comprising one of the PRMs from Table 7. Thus, a total of 58 formulations were prepared. Each sample is formulated with 0.6% of the indicated PRM on Table 7. A base formulation with no PRM was also tested for calibration. Each formulation was evaluated with test bars at 1.0% strain vs. a control with no strain using the Test Method to Determine Crazing Effect of Perfume Raw Materials and Plastic Material Under Strain described above.

TABLE 6

| | Wt % |
|---|---|
| Individual Perfume Raw Material | 0.6 |
| (PRM) Indicated per Table 7 | |
| Surfactants & Emulsifiers | 1.8 |
| Buffers & Other Stabilizers | 0.4 |
| Cyclodextrin derivative | 0.15 |
| Ethanol | 5 |
| Water | To 100 |

As demonstrated in Table 7, the perfume materials do not induce crazing when the plastic is not under strain, simulating how perfume material will act in unpressurized conditions. As seen with test bars at 1% Strain, the PRMs cause crazing at different level depending on the type of PRM.

TABLE 7

| Formu- | | Avg % | Avg % | |
|---|---|---|---|---|
| lation | PRM in Aqueous Formulation | Crazed at | Crazed at | Standard |
| No. | per Table 6 | No Strain | 1% Strain | Deviation |
| 1 | no prm | 0% | 0% | 0% |
| 2 | pyranol | 0% | 0% | 0.0% |
| 3 | silvanone m | 0% | 0% | 0.0% |
| 4 | p-cresyl methyl ether (anisole) | 0% | 0% | 0.0% |
| 5 | hexamethylindanopyran | 0% | 0% | 0.0% |
| 6 | anisic aldehyde | 0% | 0% | 0.0% |
| 7 | iso e super or wood | 0% | 2% | 4.2% |
| 8 | benzyl salicylate | 0% | 3% | 4.8% |
| 9 | benzyl alcohol | 0% | 4% | 6.2% |
| 10 | prenyl acetate | 0% | 11% | 2.9% |
| 11 | benzaldehyde | 0% | 13% | 2.9% |
| 12 | phenyl ethyl alcohol | 0% | 16% | 3.7% |
| 13 | p-methyl phenoxy acetaldehyde | 0% | 16% | 1.7% |
| 14 | ethyl butyrate | 0% | 18% | 1.1% |
| 15 | delta muscenone 962191 | 0% | 18% | 1.3% |
| 16 | habanolide 100% | 0% | 19% | 0.3% |
| 17 | benzyl acetate | 0% | 22% | 6.5% |
| 18 | diphenyloxide | 0% | 23% | 6.2% |
| 19 | helional | 0% | 24% | 5.3% |
| 20 | lyral | 0% | 25% | 5.0% |
| 21 | hexyl salicylate | 0% | 26% | 5.6% |
| 22 | ethylene brassylate | 0% | 27% | 1.9% |
| 23 | cis-3-hexenyl acetate | 0% | 28% | 4.6% |
| 24 | methyl phenyl carbinyl acetate | 0% | 30% | 6.0% |
| 25 | citronellyl oxyacetaldehyde | 0% | 35% | 1.6% |
| 26 | phenoxy ethyl iso butyrate | 0% | 35% | 8.8% |
| 27 | hexyl cinnamic aldehyde | 0% | 38% | 3.7% |
| 28 | ligustral or triplal | 0% | 38% | 2.2% |
| 29 | melonal | 0% | 38% | 1.0% |
| 30 | methyl dihydro jasmonate | 0% | 39% | 5.9% |
| 31 | delta damascone | 0% | 40% | 1.8% |
| 32 | ionone beta | 0% | 40% | 0.5% |
| 33 | floralozone | 0% | 40% | 9.6% |
| 34 | citronellol | 0% | 40% | 7.2% |
| 35 | cymal | 0% | 40% | 2.1% |
| 36 | ionone alpha | 0% | 40% | 3.2% |
| 37 | undecalactone | 0% | 40% | 3.2% |
| 38 | undecavertol | 0% | 41% | 3.7% |
| 39 | 4-tertiary butyl cyclohexyl | 0% | 42% | 0.9% |
| | acetate | | | |
| 40 | verdox | 0% | 43% | 1.6% |
| 41 | allyl caproate | 0% | 44% | 4.1% |
| 42 | ionone gamma methyl | 0% | 46% | 2.2% |
| 43 | dihydro myrcenol | 0% | 46% | 3.1% |
| 44 | dimethylbenzylcarbinylacetate | 0% | 46% | 7.9% |
| 45 | violiff | 0% | 47% | 5.0% |
| 46 | d-limonene | 0% | 47% | 2.1% |
| 47 | frutene | 0% | 48% | 1.7% |
| 48 | linalool | 0% | 48% | 1.9% |
| 49 | linalyl acetate | 0% | 50% | 4.5% |
| 50 | p.t. bucinal | 0% | 50% | 1.8% |
| 51 | cyclohexyl acetate | 0% | 53% | 2.5% |
| 52 | p-cymene | 0% | 56% | 6.3% |
| 53 | ethyl linalool | 0% | 57% | 1.4% |
| 54 | ethyl-2-methyl butyrate | 0% | 60% | 2.2% |
| 55 | iso nonyl acetate | 0% | 61% | 7.0% |
| 56 | manzanate (ethyl 2 methyl | 0% | 62% | 1.8% |
| | pentanoate, ethyl 2 methyl | | | |
| | valerate) | | | |
| 57 | sclareolate | 0% | 64% | 3.6% |
| 58 | aphermate | 0% | 64% | 4.1% |

Crazing Profiles and Critical Concentrations of Different Types of PRMs

TABLE 8

| | Weight % |
|---|---|
| PRM | As indicated per |
| | Table 9 |
| Surfactants & Emulsifiers | 1.8 |
| Buffers & Other Stabilizers | 0.4 |
| Cyclodextrin derivative | 0.15 |
| Ethanol | 5 |
| Water | To 100 |

Each PRM listed in Table 9 was tested in the aqueous perfume composition of Table 8 at 0.01%, 0.05%, 0.1%, 0.6% and 1.2% by weight of the aqueous freshening composition. A control with no PRM is also tested. All the formulations were tested at 1% Strain according to the Test Method to Determine Crazing Effect of Perfume Raw Materials and Plastic Material Under Strain described above.

TABLE 9
% Crazed of different PRMs at Different
Concentrations and at 1% Strain

| | PRM Concentration (wt. %) | | | | | | |
|---|---|---|---|---|---|---|---|
| | 0 | 0.01 | 0.05 | 0.1 | 0.3 | 0.6 | 1.2 |
| Benzaldehyde | 0% | 0% | 0% | 0% | 16% | 13% | 6% |
| Pyranol | 0% | 0% | 0% | 0% | 0% | 0% | 0% |
| Citronellol | 0% | 0% | 0% | 0% | 21% | 40% | 44% |
| 4-Tertiary Butyl | 0% | 0% | 0% | 0% | 31% | 42% | 50% |
| Cyclohexyl Acetate | | | | | | | |
| Delta Damascone | 0% | 0% | 0% | 0% | 34% | 40% | 50% |
| Cymal | 0% | 0% | 0% | 0% | 27% | 45% | 55% |
| Linalool | 0% | 0% | 0% | 0% | 24% | 48% | 60% |
| Manzanate | 0% | 0% | 0% | 0% | 27% | 62% | 74% |

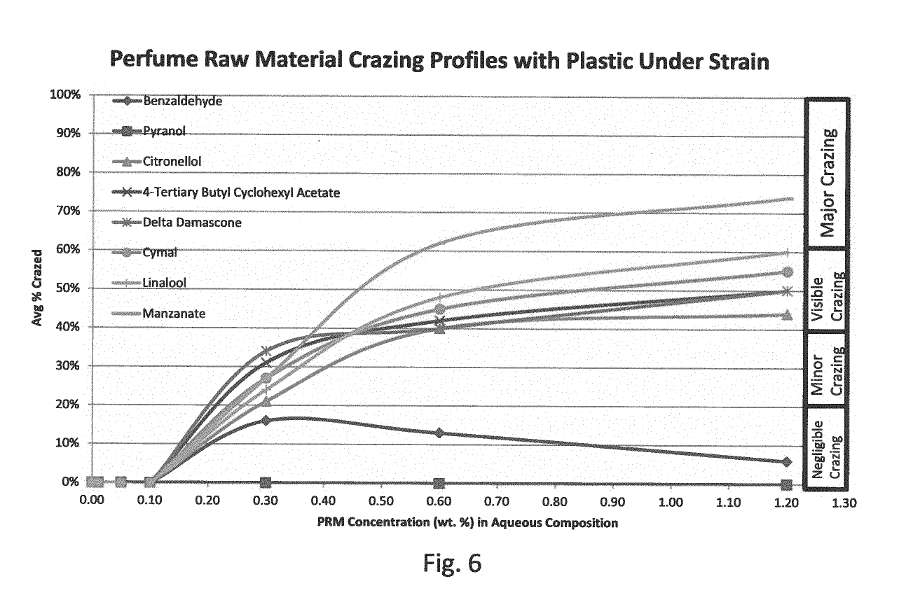

The results are reported in Table 9 above and in FIG. 6. The results in Table 9 and FIG. 6 demonstrate that crazing is initiated at certain critical concentrations (wt. %) for different types of PRMs. All of the PRMs tested show no or negligible crazing up to 0.1% in aqueous formulation. It can also be observed that crazing begins to plateau for most PRMs after a certain concentration. It is shown that Highly Compatible PRMs like benzaldehyde and pyranol exhibit no to negligible crazing at all levels tested. Controlled and Limited PRMs start to show minor crazing at about 0.3 wt % and do not reach visible Craze % (% Craze above 40%) until about 0.6 wt. %.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

It should be understood that every maximum numerical limitation given throughout this specification will include every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A freshening product comprising:
a pressurized plastic container having an internal gage pressure of about 414 kPa to about 1100 kPA; and
an aqueous freshening composition comprising:
(a) at least one controlled perfume raw material (“Controlled PRM”) selected from the group consisting of: (z)-1-((1r,2s)-2,6,6-trimethylcyclohex-3-en-1-yl)but-2-en-1-one; 3-(2-ethylphenyl)-2,2-dimethylpropanal; 3,7-dimethyloct-6-en-1-ol; 3-(4-isopropylphenyl)-2-methylpropanal; 5-heptyldihydrofuran-2(3h)-one; (e)-4-methyldec-3-en-5-ol; 4-(tert-butyl)cyclohexyl acetate; 2-(tert-butyl)cyclohexyl acetate; allyl hexanoate; 2,6-dimethyloct-7-en-2-ol; 2-methyl-1-phenylpropan-2-yl acetate; (z)-cyclooct-4-en-1-yl methyl carbonate; (r)-1-methyl-4-(prop-1-en-2-yl)cyclohex-1-ene; 3,7-dimethylocta-1,6-dien-3-ol; 3,7-dimethylocta-1,6-dien-3-yl acetate; 3-(4-(tert-butyl)phenyl)-2-methylpropanal; cyclohexyl acetate; p-cymene; (e)-3,7-dimethylnona-1,6-dien-3-ol; (e)-4-(2,6,6-trimethylcyclohex-1-en-1-yl)but-3-en-2-one; (e)-4-(2,6,6-trimethylcyclohex-2-en-1-yl)but-3-en-2-one; (z)-3-methyl-4-(2,6,6-trimethylcyclohex-2-en-1-yl)but-3-en-2-one; 1-(2,6,6-trimethyl-1-cyclohex-2-en-1-yl)pent-1-en-3-one; 3a,4,5,6,7,7a-hexahydro-4,7-methano-1(3)h-inden-1-yl propanoate; 3a,4,5,6,7,7a-hexahydro-4,7-methano-1H-inden-6-yl propionate; 3a,4,5,6,7,7a-hexahydro-1H-4,7-methanoinden-6-yl acetate; 3a,4,5,6,7,7a-hexahydro-1H-4,7-methanoinden-1-yl acetate; 3a,4,5,6,7,7a-hexahydro-4,7-methano-1H-inden-5-yl acetate; ethyl 2-(2-methyl-1,3-dioxolan-2-yl)acetate; and combinations thereof; or
(b) at least one limited perfume raw material (“Limited PRM”) selected from the group consisting of: ethyl 2-methylbutanoate; 3,5,5-trimethylhexyl acetate; ethyl 2-methylpentanoate; propyl (s)-2-(tert-pentyloxy)propanoate; 1-(3,3-dimethylcyclohexyl)ethyl formate, 4-methylquinoline; 1-heptanol; and combinations thereof,
wherein each Controlled PRM or each Limited PRM, when present, is present at a level up to 0.3%, by weight of the aqueous freshening composition.

2. The freshening product of claim 1, wherein the total level of Controlled PRMs present in the aqueous freshening composition is 0.6 wt. % or less, by weight of the aqueous freshening composition, and wherein the total level of Limited PRMs present in the aqueous freshening composition is 0.1 wt. % or less, by weight of the aqueous freshening composition.

3. The freshening product of claim 1, wherein the aqueous freshening composition comprises at least one moderately compatible perfume raw material “(Moderately Compatible “PRM”) selected from the group consisting of: benzyl acetate; oxydibenzene; 3-(benzo[d][1,3]dioxol-5-yl)-2-methylpropanal; 4-(4-hydroxy-4-methylpentyl)cyclohex-3-ene-1-carbaldehyde; hexyl 2-hydroxybenzoate; 1,4-dioxacycloheptadecane-5,17-dione; (Z)-hex-3-en-1-yl acetate; 1-phenylethyl acetate; 2-((3,7-dimethyloct-6-en-1-yl)oxy)acetaldehyde; 2-phenoxyethyl isobutyrate; (Z)-2-benzylideneoctanal; 2,4-dimethylcyclohex-3-ene-1-carbaldehyde; 2,6-dimethylhept-5-enal; methyl 2-(3-oxo-2-pentylcyclopentyl)acetate; benzyl methyl ether; benzyl ether; 4-methylbenzaldehyde; 3,5,5-trimethyl-2-cyclohexen-1-one; and combinations thereof, wherein each Moderately Compatible PRM, when present, is present at level up to 1.0 wt. %, by weight of the aqueous freshening composition.

4. The freshening product of claim 1, wherein the pressurized plastic container comprises a hoop tensile strain of about 0.1% to about 2%.

5. The freshening product of claim 1, wherein the aqueous carrier is present in an amount greater than 70 wt, %, by weight of said composition.

6. The freshening product of claim 1, wherein the perfume mixture is present in an amount of about 0.01 wt. % to about 10 wt. %, by weight of said aqueous freshening composition.

7. The freshening product of claim 1, further comprising a propellant selected from the group consisting of: nitrogen, carbon dioxide, compressed air, and mixtures thereof.

8. The freshening product of claim 1, wherein the pressurized plastic container comprises a bag-in-bottle container comprising a hydrofluoro olefin propellant.

9. The freshening product of claim 7, wherein the propellant is free of hydrocarbons.

10. The freshening product of claim 1, further comprising 1-((2S,3S)-2,3,8,8-tetramethyl-1,2,3,4,5,6,7,8-octahydronaphthalen-2-yl)ethan-1-one; or benzyl 2-hydroxybenzoate.

11. A freshening product comprising:
a pressurized plastic container; and
an aqueous freshening composition comprising at least one highly compatible perfume raw material (“Highly Compatible PRM”) selected from the group consisting of: 2-isobutyl-4-methyltetrahydro-2H-pyran-4-ol; (3aR,5aR,9aR,9bR)-3a,6,6,9a-tetramethyldodecahydronaphtho[2,1-b]furan; 1-methoxy-4-methylbenzene; 4,6,6,7,8,8-hexamethyl-1,3,4,6,7,8-hexahydrocyclopenta[g]isochromene; 4-methoxybenzaldehyde; phenylmethanol; 3-methylbut-2-en-1-yl acetate; benzaldehyde; 2-phenylethan-1-ol; 2-(p-tolyloxy)acetaldehyde; ethyl butyrate; (E)-3-methylcyclopentadec-4-en-1-one; (E)-oxacyclohexadec-13-en-2-one, and combinations thereof.

12. The freshening product of claim 11, wherein the pressurized plastic container is pressurized to an internal gage pressure of about 414 kPa to about 1100 kPA.

13. The freshening product of claim 11, wherein the aqueous freshening composition comprises at least two Highly Compatible PRMs.

14. The freshening product of claim 11, wherein the aqueous freshening composition comprises at least one moderately compatible perfume raw material “(Moderately Compatible “PRM”) selected from the group consisting of: benzyl acetate; oxydibenzene; 3-(benzo[d][1,3]dioxol-5-yl)-2-methylpropanal; 4-(4-hydroxy-4-methylpentyl)cyclohex-3-ene-1-carbaldehyde; hexyl 2-hydroxybenzoate; 1,4-dioxacycloheptadecane-5,17-dione; (Z)-hex-3-en-1-yl acetate; 1-phenylethyl acetate; 2-((3,7-dimethyloct-6-en-1-yl)oxy)acetaldehyde; 2-phenoxyethyl isobutyrate; (Z)-2-benzylideneoctanal; 2,4-dimethylcyclohex-3-ene-1-carbaldehyde; 2,6-dimethylhept-5-enal; methyl 2-(3-oxo-2-pentylcyclopentyl)acetate, benzyl methyl ether; benzyl ether; 4-methylbenzaldehyde; 3,5,5-trimethyl-2-cyclohexen-1-one; and combinations thereof, wherein each Moderately Compatible PRM, when present, is present at level up to 1.0 wt. %, by weight of the aqueous freshening composition.

15. The freshening product of claim 14, wherein the total level of Moderately Compatible PRMs present in the aqueous freshening composition is 1.2 wt. % or less, by weight of the aqueous freshening composition.

16. The freshening product of claim 11, wherein the aqueous freshening composition comprises at least one controlled perfume raw material (“Controlled PRM”) selected from the group consisting of: (z)-1-((1r,2s)-2,6,6-trimethylcyclohex-3-en-1-yl)but-2-en-1-one; 3-(2-ethylphenyl)-2,2-dimethylpropanal; 3,7-dimethyloct-6-en-1-ol; 3-(4-isopropylphenyl)-2-methylpropanal; 5-heptyldihydrofuran-2(3h)-one; (e)-4-methyldec-3-en-5-ol; 4-(tert-butyl)cyclohexyl acetate; 2-(tert-butyl)cyclohexyl acetate; allyl hexanoate; 2,6-dimethyloct-7-en-2-ol; 2-methyl-1-phenylpropan-2-yl acetate; (z)-cyclooct-4-en-1-yl methyl carbonate; (r)-1-methyl-4-(prop-1-en-2-yl)cyclohex-1-ene; 3,7-dimethylocta-1,6-dien-3-ol; 3,7-dimethylocta-1,6-dien-3-yl acetate; 3-(4-(tert-butyl)phenyl)-2-methylpropanal; cyclohexyl acetate; p-cymene; (e)-3,7-dimethylnona-1,6-dien-3-ol; (e)-4-(2,6,6-trimethylcyclohex-1-en-1-yl)but-3-en-2-one; (e)-4-(2,6,6-trimethylcyclohex-2-en-1-yl)but-3-en-2-one; (z)-3-methyl-4-(2,6,6-trimethylcyclohex-2-en-1-yl)but-3-en-2-one; 1-(2,6,6-trimethyl-1-cyclohex-2-en-1-yl)pent-1-en-3-one; 3a,4,5,6,7,7a-hexahydro-4,7-methano-1(3)h-inden-1-yl propanoate; 3a,4,5,6,7,7a-hexahydro-4,7-methano-1H-inden-6-yl propionate; 3a,4,5,6,7,7a-hexahydro-1H-4,7-methanoinden-6-yl acetate; 3a,4,5,6,7,7a-hexahydro-1H-4,7-methanoinden-1-yl acetate; 3a,4,5,6,7,7a-hexahydro-4,7-methano-1H-inden-5-yl acetate; ethyl 2-(2-methyl-1,3-dioxolan-2-yl)acetate; and combinations thereof, wherein each Controlled PRM, when present, is present at level up to 0.3 wt. %, by weight of the aqueous freshening composition.

17. The freshening product of claim 16, wherein the total level of Controlled PRMs present in the aqueous freshening composition is 0.6 wt. % or less, by weight of the aqueous freshening composition.

18. The freshening product of claim 11, wherein the aqueous freshening composition comprises at least one limited perfume raw material (“Limited PRM”) selected from the group consisting of: ethyl 2-methylbutanoate; 3,5,5-trimethylhexyl acetate; ethyl 2-methylpentanoate; propyl (s)-2-(tert-pentyloxy)propanoate; 1-(3,3-dimethylcyclohexyl)ethyl formate, 4-methylquinoline; 1-heptanol; and combinations thereof, wherein each Limited PRM, when present, is present at level up to 0.3 wt. %, by weight of the aqueous freshening composition.

19. The freshening product of claim 18, wherein the total level of Limited PRMs present in the aqueous freshening composition is 0.1 wt. % or less, by weight of the aqueous freshening composition.

20. The freshening product of claim 11, wherein the pressurized plastic container comprises a hoop tensile strain of about 0.1% to about 2%.

21. The freshening product of claim 11, wherein the aqueous carrier is present in an amount greater than 70 wt, %, by weight of said composition.

22. The freshening product of claim 11, further comprising a propellant selected from the group consisting of: nitrogen, carbon dioxide, compressed air, and mixtures thereof.

23. The freshening product of claim 11, wherein the pressurized plastic container comprises a bag-in-bottle container comprising a hydrofluoro olefin propellant.

24. The freshening product of claim 22, wherein said propellant is free of hydrocarbons.

25. The freshening product of claim 10, further comprising 1-((2S,3S)-2,3,8,8-tetramethyl-1,2,3,4,5,6,7,8-octahydronaphthalen-2-yl)ethan-1-one; or benzyl 2-hydroxybenzoate.
